(19) United States
(12) Patent Application Publication
(10) Pub. No.: US 2025/0223544 A1
(43) Pub. Date: Jul. 10, 2025

(54) SYNTHETIC QUORUM-REGULATED LYSIS

(71) Applicant: The Regents of the University of California, Oakland, CA (US)

(72) Inventors: Jeff Hasty, Encinitas, CA (US); Lev Tsimring, San Diego, CA (US); Spencer R. Scott, Rancho Santa Fe, CA (US); Muhammad Omar Din, San Diego, CA (US)

(21) Appl. No.: 18/926,545

(22) Filed: Oct. 25, 2024

Related U.S. Application Data
(63) Continuation of application No. 18/072,256, filed on Nov. 30, 2022, now abandoned.
Application No. 18/072,256 is a continuation of application No. 16/614,613, filed on Nov. 18, 2019, now abandoned.
(60) Provisional application No. 62/508,801, filed on May 19, 2017.

(51) Int. Cl. C12N 1/20; C12M 3/06; C12R 1/19; C12R 1/42

(52) U.S. Cl. CPC C12N 1/20; C12M 23/16; C12R 2001/19; C12R 2001/42

(57) ABSTRACT

Provided are bacterial strains, methods of culturing bacterial cells using synthetic quorum-regulated lysis, and uses thereof. For example, the present disclosure describes methods of maintaining a co-culture by quorum sensing. These methods comprise co-culturing at least a first bacterial strain and a second bacterial strain during a period of time of at least 12 hours.

### CLAIM OF PRIORITY

This application is a continuation of U.S. patent application Ser. No. 18/072,256, filed on Nov. 30, 2022, which is a continuation of U.S. patent application Ser. No. 16/614,613, filed Nov. 18, 2019, which is a 35 U.S.C. 371 U.S. National Phase Application of PCT/US2018/033555, filed on May 18, 2018, which claims the benefit of U.S. Provisional Patent Application Ser. No. 62/508,801, filed on May 19, 2017. The entire contents of the foregoing are hereby incorporated by reference.

### FEDERALLY SPONSORED RESEARCH OR DEVELOPMENT

This invention was made with Government support under GM069811, and GM085764 awarded by the National Institutes of Health. The government has certain right in the invention.

### TECHNICAL FIELD

This disclosure relates to methods of culturing bacterial cells using synthetic quorum-regulated lysis, and more particularly to a co-lysis system. This invention also relates to uses of synthetic synchronized lysis circuits.

### BACKGROUND

Microbial ecologists are increasingly turning to small, synthesized ecosystems¹⁻⁵ as a reductionist tool to probe the complexity of native microbiomes^{6,7}. Concurrently, synthetic biologists have gone from single-cell gene circuits⁸⁻¹¹ to controlling whole populations using intercellular signaling¹²⁻¹⁶.

### SUMMARY

Provided herein are methods of co-culturing by quorum sensing, bacterial strains useful in co-culture systems and methods, co-culture systems, and pharmaceutical compositions, drug delivery systems, and methods of treating disease related thereto. In some embodiments, the co-lysis systems are provided that operate in an orthogonal or essentially orthogonal manner.

In some embodiments, methods of maintaining a co-culture by quorum sensing are provided that include: co-culturing at least two (e.g., 2, 3, 4, 5, 6, 7, 8, 9, 10) bacterial strains at certain ratios (e.g., 1:1000, 1:900, 1:800, 1:750, 1:700, 1:650, 1:600, 1:550, 1:500, 1:450, 1:400, 1:350, 1:300, 1:250, 1:200, 1:150, 1:100, 1:90, 1:80, 1:70, 1:60, 1:50, 1:40, 1:30, 1:20, 1:10, 1:9, 1:8, 1:1:7, 1:6, 1:5, 1:4, 1:3, 1:2, 1:1) during a period of time (e.g., at least 12 hours, at least 24 hours, at least 48 hours, at least 72 hours, at least 96 hours; or 12, 24, 48, 72, 96, or more hours; or 1, 2, 3, 4, 5, 6, 7, 8, 9, 10 or more days); wherein: at least one of the at least two bacterial strains has a growth advantage compared to at least one other bacterial strain; at least one of the at least two bacterial strains comprises a lysis plasmid and an activator plasmid.

In some embodiments, the at least two bacterial strains include a first bacterial strain and a second bacterial strain. In some embodiments, each of the first and second bacterial strains comprises a lysis plasmid having a lysis gene under the control of an activatable promoter; and an activator plasmid having an activator gene, the expression of which promotes the accumulation of a quorum sensing molecule, wherein both the activatable promoter of the lysis gene and the expression of the activator gene is activated by the quorum sensing molecule, wherein the quorum-sensing molecule of the first strain is different from the quorum-sensing molecule of the second strain, and wherein each quorum-sensing molecule of the first and second strains has no or substantially no effect on the activatable promoter of the lysis gene of the other strain.

As used herein, “substantially no effect” means no measurable effect on the activatable promoter, as measured by the expression of the activatable promotor of a fluorescent protein.

In some embodiments, the at least two bacterial strains (e.g., at least a first bacterial strain and a second bacterial strain) are metabolically competitive. In some embodiments, at least one of the at least two bacterial strains (e.g., at least one of a first bacterial strain and a second bacterial strain) are E. coli, S. typhimurium, or a bacterial variant thereof. In some embodiments, at least one of the at least two bacterial strains (e.g., at least one of a first bacterial strain and a second bacterial strain) are Gram-negative bacterial strains, e.g., a Salmonella strain, an Acetobacter strain, an Enterobacter strain, a Fusobacterium strain, a Helicobacter strain, a Klebsiella strain, or an E. coli strain. In some embodiments, the at least two bacterial strains (e.g., at least a first bacterial strain and a second bacterial strain) are Gram-positive bacterial strain, e.g., a Actinomyces strain, a Bacillus strain, a Clostridium strain, an Enterococcus strain, or a Lactobacillus strain. In some embodiments, the at least two bacterial strains are both Gram negative bacterial strains or both Gram positive strains. In some embodiments, at least one of the at least two bacterial strains (e.g., at least a first bacterial strain and a second bacterial strain) is a Gram negative bacterial strain. In some embodiments, at least one of the at least two bacterial strains (e.g., at least a first bacterial strain and a second bacterial strain) is a Gram positive bacterial strain. In some embodiments, at least one of the at least two bacterial strains (e.g., at least a first bacterial strain and a second bacterial strain) comprising the lysis plasmid and the activator plasmid does not have a growth advantage compared to at least one other bacterial strain.

In some embodiments, the lysis plasmid comprises a lysis gene, an activatable promoter, and optionally a reporter gene; and the activator plasmid comprises an activator gene, a degradation tag, and optionally a reporter gene. In some embodiments, the lysis gene is E from a bacteriophage ΦX174. In some embodiments, the activatable promoter is a LuxR-N-acyl homoserine lactone (AHL) activatable luxI promoter and the activator gene is a LuxI. In some embodiments, the activatable promoter is a RpaR-N-acyl homoserine lactone (AHL) activatable RpaI promoter and the activator gene is a RpaI. In some embodiments, the reporter gene is green fluorescent protein (GFP), cyan fluorescent protein (CFP), red fluorescent protein (RFP) or a variant thereof. In some embodiments, the degradation tag is an ssrA-LAA degradation tag. In some embodiments, each of the at least two bacterial stains comprises the lysis plasmid and the activator plasmid. In some embodiments, each of the at least two bacterial strains (e.g., each of at least a first bacterial strain and a second bacterial strain) comprises a different reporter gene.

In some embodiments, the co-culture is inoculated at a ratio of 1:1000 (e.g., 1:900, 1:800, 1:750, 1:700, 1:650, 1:600, 1:550, 1:500, 1:450, 1:400, 1:350, 1:300, 1:250, 1:200, 1:150, 1:100, 1:90, 1:80, 1:70, 1:60, 1:50, 1:40, 1:30, 1:20, 1:10, 1:9, 1:8, 1:1:7, 1:6, 1:5, 1:4, 1:3, 1:2, 1:1) of the bacterial strain having the growth advantage compared to the other bacterial strain.

In some embodiments, the plasmid is integrated into a genome of at least one of the at least two bacterial strains (e.g., at least a first bacterial strain and a second bacterial strain). In some embodiments, the plasmid further comprises a plasmid-stabilizing element. In some embodiments, the plasmid-stabilizing element is a toxin/antitoxin system or an actin-like protein partitioning system.

In some embodiments, the co-culturing occurs in a microfluidic device. In some embodiments, the co-culturing occurs in a cell culture vessel (e.g., a cell culture plate, a bioreactor).

In some embodiments, the period of time is 0 to 72 hours (e.g., o to 72; 0 to 60 hours; 0 to 48 hours; 0 to 36 hours; 0 to 24 hours; 0 to 16 hours; 0 to 14 hours; 0 to 12 hours; 0 to 10 hours; 0 to 8 hours; 0 to 6 hours; 0 to 4 hours; 0 to 2 hours; 2 to 72 hours; 2 to 60 hours; 2 to 48 hours; 2 to 36 hours; 2 to 24 hours; 2 to 16 hours; 2 to 14 hours; 2 to 12 hours; 2 to 10 hours; 2 to 8 hours; 2 to 6 hours; 2 to 4 hours; 4 to 72 hours; 4 to 60 hours; 4 to 48 hours; 4 to 36 hours; 4 to 24 hours; 4 to 16 hours; 4 to 14 hours; 4 to 12 hours; 4 to 10 hours; 4 to 8 hours; 4 to 6 hours; 6 to 8 hours; 6 to 10 hours; 6 to 12 hours; 6 to 14 hours; 6 to 16 hours; 6 to 18 hours; 6 to 20 hours; 6 to 22 hours 6 to 24 hours; 8 to 10 hours; 8 to 12 hours; 8 to 16 hours; 8 to 24 hours; 8 to 36 hours; 8 to 48 hours; 8 to 60 hours; 8 to 72 hours; 1 to 2 hours; 1 to 3 hours; 1 to 4 hours; 1 to 6 hours; 1 to 8 hours; 1 to 10 hours; 1 to 12 hours; 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57,58, 59, 60, 61, 62, 63, 64, 65, 66, 67, 68, 69, 70, 71 or 72 hours).

In some embodiments, the co-culturing of the at least two bacterial strains (e.g., at least a first bacterial strain and a second bacterial strain) is in a constant lysis state; wherein the constant lysis state is characterized by a steady-state balance of growth and lysis of the at least two bacterial strains (e.g., at least a first bacterial strain and a second bacterial strain). In some embodiments, the co-culturing of the at least two bacterial strains (e.g., at least a first bacterial strain and a second bacterial strain) is oscillatory; wherein the oscillatory co-culturing indicates a high level of activator degradation in at least one of the two bacterial strains (e.g., at least a first bacterial strain and/or a second bacterial strain).

Provided herein are bacterial strains including a lysis plasmid and an activator plasmid; wherein the lysis plasmid comprises a lysis gene, an activatable promoter, and optionally a reporter gene; and the activator plasmid comprises an activator gene, a degradation tag, and optionally a reporter gene.

In some embodiments, the lysis gene is E from a bacteriophage ΦX174.

In some embodiments, the activatable promoter is a LuxR-N-acyl homoserine lactone (AHL) activatable luxI promoter and the activator gene is a LuxI. In some embodiments, the activatable promoter is a RpaR-N-acyl homoserine lactone (AHL) activatable RpaI promoter and the activator gene is a RpaI.

Also provided herein are pharmaceutical composition that include any of the bacterial strains described herein. In some embodiments, the pharmaceutical composition is formulated for in situ drug delivery. A pharmaceutical composition is formulated to be compatible with its intended route of administration. Examples of routes of administration include oral or parenteral, e.g., intravenous, intradermal, subcutaneous, inhalation, transdermal (topical), transmucosal, and rectal administration. As used herein the language “pharmaceutically acceptable carrier” includes solvents, dispersion media, coatings, isotonic and absorption delaying agents, and the like, compatible with pharmaceutical administration. Supplementary active compounds can also be incorporated into the compositions.

Provided herein are systems including: a co-culture of at least two bacterial strains (e.g., at least a first bacterial strain and a second bacterial strain), wherein the at least two bacterial strains comprise a first bacterial strain having at least a portion of a first synchronized lysis circuit, wherein the first synchronized lysis circuit comprises a first lysis plasmid and a first activator plasmid and wherein the first lysis plasmid is activated by the first activator plasmid. In some embodiments, the first bacterial strain comprises the first lysis plasmid. In some embodiments, the first bacterial strain comprises the first activator plasmid. In some embodiments, the at least two bacterial strains further comprise a second bacterial strain. In some embodiments, the second bacterial strain comprises the first activator plasmid. In some embodiments, each of the first bacterial strain and the second bacterial strain comprise the first activator plasmid.

In some embodiments, the first lysis plasmid of the first bacterial strain operates independent of at least one other bacterial strain in the co-culture. In some embodiments, the first lysis plasmid of the first bacterial strain responds to a signal generated by at least one other bacterial strain in the co-culture.

In some embodiments, the second bacterial strain has at least a portion of a second synchronized lysis circuit, wherein the second synchronized lysis circuit comprises a second lysis plasmid and a second activator plasmid. In some embodiments, the second bacterial strain comprises the second lysis plasmid. In some embodiments, the second bacterial strain comprises the second activator plasmid. In some embodiments, the first bacterial strain comprises the second activator plasmid. In some embodiments, the second lysis plasmid of the second bacterial strain operates independent of at least the first bacterial strain. In some embodiments, the second lysis plasmid of the second bacterial strain responds to a signal generated by the first bacterial strain.

In some embodiments of any of the systems described herein, the signal is a quorum sensing signal. In some embodiments, the first activator plasmid encodes a quorum sensing signal. In some embodiments, the second activator plasmid encodes a quorum sensing signal.

In some embodiments, at least one of the at least two bacterial strains (e.g., at least one of a first bacterial strain and a second bacterial strain) has a growth advantage compared to at least one other bacterial strain. In some embodiments, the first bacterial strain is competitive with at least one other bacterial strain in the co-culture. In some embodiments, the co-culture is stable for at least 48 hours.

In some embodiments, the at least two bacterial strains (e.g., at least a first bacterial strain and a second bacterial strain) do not comprise engineered positive or negative interactions between each other. In some embodiments, at least one of the at least two bacterial strains (e.g., at least one of a first bacterial strain and a second bacterial strain) dynamically controls its population without exogenous input. In some embodiments, each of at least two of the at least two bacterial strains (e.g., each of at least a first bacterial strain and a second bacterial strain) dynamically controls its own population without exogenous input. In some embodiments, the system further comprises one or more plasmid stabilizing elements.

In some embodiments, the plasmid stabilizing element is selected from a toxin/antitoxin system and an actin-like protein partitioning system.

In some embodiments, the first activator plasmid encodes a degradation tagging sequence. In some embodiments, the second activator plasmid encodes a degradation tagging sequence. In some embodiments, the first activator plasmid encodes an N-acyl homoserine lactone.

Provided herein are drug delivery systems including any of the systems described herein. Provided herein are periodic drug delivery systems including any of the systems described herein.

Provided herein are microfluidic sample traps including any of the systems described herein.

Provided herein are microfluidic devices including one or more microfluidic sample traps. In some embodiments, the microfluidic system further includes at least one channel in fluid communication with the microfluidic sample trap.

In one aspect, provided herein is a method of maintaining a co-culture by quorum sensing, the method comprising co-culturing at least a first bacterial strain and a second bacterial strain during a period of time of at least 12 hours; wherein at least one of the first and second bacterial strains has a growth advantage compared to at least one other bacterial strain; and each of the first and second bacterial strains comprises: a lysis plasmid having a lysis gene under the control of an activatable promoter; and an activator plasmid having an activator gene, the expression of which promotes the accumulation of a quorum sensing molecule, wherein both the activatable promoter of the lysis gene and the expression of the activator gene is activated by the quorum sensing molecule, wherein the quorum-sensing molecule of the first strain is different from the quorum-sensing molecule of the second strain, and wherein each quorum-sensing molecule of the first and second strains has no or substantially no effect on the activatable promoter of the lysis gene of the other strain.

In another aspect, a bacterial strain is provided comprising a lysis plasmid and an activator plasmid; wherein the lysis plasmid comprises a lysis gene, an activatable promoter, and optionally a reporter gene; and the activator plasmid comprises an activator gene, a degradation tag, and optionally a reporter gene.

In another aspect, a pharmaceutical composition is provided comprising any of the bacterial strains described herein.

In another aspect, a system is provided comprising a co-culture of at least a first bacterial strain and a second bacterial strain, wherein the first bacterial strain has at least a portion of a first synchronized lysis circuit, wherein the first synchronized lysis circuit comprises a first lysis plasmid, a first activator plasmid, and a first plasmid stabilizing element, and wherein the first lysis plasmid is activated by the first activator plasmid, and wherein the second bacterial strain has at least a portion of a second synchronized lysis circuit, wherein the second synchronized lysis circuit comprises a second lysis plasmid, a second activator plasmid, and a second plasmid stabilizing element, and wherein the second lysis plasmid is activated by the second activator plasmid, and wherein the first and second synchronized lysis circuits are orthogonal in that each has no or substantially no effect upon the other.

In another aspect, a drug delivery system is provided comprising the system of any one of the systems described herein.

In another aspect, a periodic drug delivery system is provided comprising any one of the systems described herein.

In another aspect, a method of treating a disease in a subject is provided, the method comprising administering to a subject in need therapeutically effective amounts of any of the bacterial strains described herein or any of the pharmaceutical compositions described herein, to thereby treat the disease in the subject.

The systems, methods, and compositions described herein provide several advantages. Synthetic biologists have used lysis to control populations before¹², but not until recently have populations been engineered to dynamically control their own population without exogenous input¹⁶. Since the lysis systems rely on DNA parts carried on plasmids, undesired mutations may arise which can hinder the function of the circuit. Bacteria may mutate toxic or burdensome genes, and any possible mutants may gain a selective advantage over non-mutated members of the population. In this regard, strategies to enhance stability of the circuit components inside the host cells could help ensure long term robustness of the synthetic ecosystem²⁴. Additionally, in the absence of antibiotics, bacteria may encounter a selective pressure to lose the circuit plasmids. This may result in difficulties when introducing the synthetic ecosystem to an environment without any selective agents. Some ways to address these challenges include integrating circuit components within the genome or using plasmid-stabilizing elements in the circuit. Elements such as toxin/antitoxin systems and actin-like protein partitioning systems have previously been shown to stabilize plasmids in environments without antibiotics²⁵. The emergence of escapees is a direct consequence of strong selection imposed by periodic lysis, and recent evidence also suggests that repeated pruning of a population suppresses beneficial mutations that confer growth advantages unrelated to the lysis circuit²⁶. Therefore, the strategies described herein (e.g., ortholysis strategy, or orthogonal co-lysis) are attractive methodologies to impose certain population dynamics or types of selection in evolution experiments. The challenge in maintaining a population of metabolically competitive microbial organisms has long been recognized²¹. Strategies to maintain the long-term stability of engineered microbial ecosystems that have thus far been investigated mainly consist of mutualistic interactions, such as metabolic interdependencies, or predator-prey type interactions^{27,28}. Recent evidence suggests, however, that competition is likely the dominant interaction in microbial communities²⁹. In this vein, the systems, methods, and compositions described herein (e.g., “ortholysis” or orthogonal co-lysis systems, methods, and compositions) can be viewed as a strategy to stabilize competitive strains without engineering positive and negative interactions between members of the population. Moreover, recent evidence has identified quorum-sensing controlled self-lysis as a naturally occurring phenomenon in Pseudomonas aeruginosa³⁰, which is a relevant example of how the interests of synthetic biologists and microbial ecologists are merging in the field of engineered microbial ecosystems. With the additional modeling of the circuit it becomes clear that the transition from monoculture synthetic biology to synthetic engineered ecosystems will be marked by an explosion of possibilities. A circuit designed for monocultures, such as the SLC, can have drastically broadened use-cases when expanded into the setting of a community. The systems, methods, and compositions described herein (e.g., “ortholysis” or orthogonal co-lysis systems, methods, and compositions) are immediately applicable for further expansion on the periodic in situ drug delivery system¹⁶. Additionally, this phenomenon of stably co-culturing two metabolically competitive strains through orthogonal self-lysing offers the possibility of many unique applications beyond drug delivery where the use of synthetic microbial ecosystems is advantageous.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1A shows a genetic diagram of a synchronized lysis circuit (SLC). The circuit contains a lysis plasmid and an activator/reporter plasmid. Transient production of LuxI eventually leads to an accumulation of N-acyl homoserine lactone (AHL) above the quorum threshold needed to activate LuxR, which begins a positive feedback loop by driving transcription off the PluxI promoters that control production LuxI, GFP, and the lysis gene ΦX174E. LuxR in this system is driven by the native luxR promoter.

FIG. 1B is a graph showing bifurcation to oscillations in a deterministic model of the lysis circuit. Ignoring initial transient behavior, minimum, maximum, and mean population density over time were determined for each parameter value. Lower α_{q} corresponds to stronger degradation.

FIG. 1C shows video stills showing bacteria harboring the SLC with strong degradation of the LuxI activator (LuxI-LAA) exhibiting oscillations in a microfluidic growth chamber. Oscillations result from repeated cycles of growth, quorum threshold reached, and self-limitation by lysis activation.

FIG. 1D shows video stills depicting bacteria harboring the SLC with weaker degradation of LuxI (LuxI with no degradation tag) exhibiting constant lysis. Constant activation of the lysis circuit results in the continual activation of GFP as well as continuous growth and lysis events within the microfluidic chamber.

FIG. 1E is a graph showing fluorescence (light grey) and population (black) over time for cells harboring the SLC with α_{q}=0.4 for the deterministic model, as seen in FIG. 1C.

FIG. 1F is a graph showing fluorescence (light grey) and cells (black) with a LAA-tagged LuxI over time.

FIG. 1G is a graph showing fluorescence (light grey) and population (black) over time for cells harboring the SLC with α_{q}=1.1 for the deterministic model

FIG. 1H is a graph showing fluorescence (light grey) and cells (black) with a TS-LAA-tagged LuxI over time.

FIG. 1I is a graph showing fluorescence (light grey) and population (black) over time for cells harboring the SLC with aq=2 for the deterministic model.

FIG. 1J is a graph showing fluorescence (light grey) and cells (black) with untagged LuxI.

FIG. 2A shows a genetic diagram of a two-strain ecosystem of self-lysing Salmonella constructed with two signal orthogonal quorum sensing systems, rpa and lux.

FIG. 2B is a graph showing batch culture growth curves of the Lux-CFP strain alone, Rpa-GFP strain alone, a 1:1 mixture, and a 1:100 (Rpa-GFP:Lux-CFP) mixture, both without the lysis gene (top) and with the lysis gene (bottom). All strains were started from the same diluted density and under the same growth conditions. Width of lines represent s.d. (n=3)

FIG. 2C is a graph showing batch culture population estimates of Lux-CFP and Rpa-GFP co-cultures. Rpa-GFP population estimated as GFP fluorescence (integrated over the full length of the experiment) of the mixture normalized by the time-integrated GFP fluorescence of Rpa-GFP cells alone. Lux-CFP population estimated as CFP fluorescence (integrated over the full length of the experiment) of the mixture normalized by the time-integrated CFP fluorescence Lux-CFP cells alone. Error bars represent standard deviation (s.d). (n=3)

FIG. 2D shows video stills of a representative co-culture of non-lysing Lux-CFP and Rpa-GFP strains showing the eventual takeover by the green strain.

FIG. 2E shows video stills of a representative co-culture of the Lux-CFP and Rpa-GFP strains with the lysis plasmid. The addition of the lysis plasmid prevents either strain from taking over for the duration of the experiment.

FIG. 2F is a graph showing a time trace of the GFP and CFP Fluorescence of the trap in the video shown in FIG. 2D.

FIG. 2G is a graph showing a time trace of the GFP and CFP Fluorescence of the trap in the video shown in FIG. 2E.

FIG. 2H is a graph showing the length of co-culture for each of the sixty traps containing the non-lysing strains.

FIG. 2I is a graph showing the length of co-culture for each of the sixty traps containing the strains with the lysis plasmids.

FIG. 3A shows video stills of a representative, virtual co-culture of two self-lysing strains both in the oscillatory regime of the lysis circuit in a simulated trap of size 60. Scale bar at top, right of micrograph indicates half of the size of the trap. Number at the bottom of the micrographs indicate iteration “Time”.

FIG. 3B shows video Stills of representative, model-generated video recreating experimental dynamics. Number at the bottom of the micrographs indicate iteration “Time”.

FIG. 3C is a graph showing a time trace of the GFP (light grey) and CFP (black) “Fluorescence” of the trap in FIG. 3A over time.

FIG. 3D is a graph showing a time trace of the GFP (light grey) and CFP (black) “Fluorescence” of the trap in FIG. 3B, as well as population of the “Lux-CFP” strain (dashed line).

FIG. 3E shows four graphsfrom left to right: (1) light grey in constant lysis phase, black in the oscillatory phase in a trap with size 20. (2) light grey in constant lysis phase, black in the oscillatory phase in a trap with size 40. (3) light grey in constant lysis phase, black in the oscillatory phase in a trap with size 60. Video in B is in this size trap with these lysing conditions. (4) Both strains in oscillatory phase with trap size 60. Video in A is in this size trap with these lysing conditions.

FIG. 4A shows a prediction of synchronized lysis circuit dynamics in a dual strain population using various communication motifs. Model-generated heat maps depicting time-averaged population ratio of light grey and black non-lysing strains in a well-mixed, constant flow co-culture, as function of “light grey”'s growth rate α₁ against “black”'s growth rate α₂. On the left of each heat-map is the communication motif it exhibits and experimental candidate QS systems to achieve the desired signaling characteristic. These traits determine the behavior and composition of the co-culture. The white dot on the heat map indicates the growth rate parameters selected for the time-series plots. Time series plots show population of the two strains as a function of time.

FIG. 4B shows a prediction of synchronized lysis circuit dynamics in a dual strain population using various communication motifs. Model-generated heat maps depicting time-averaged population ratio of light grey non-lysing strain and black lysing strain in a well-mixed, constant flow co-culture, as function of light grey's growth rate α₁ against black's growth rate α₂. On the left of each heat-map is the communication motif it exhibits and experimental candidate QS systems to achieve the desired signaling characteristic. These traits determine the behavior and composition of the co-culture. The white dot on the heat map indicates the growth rate parameters selected for the time-series plots. Time series plots show population of the two strains as a function of time. White, dashed lines indicate the growth rate at which one strain's growth rate exceeds that of the other one even for maximum lysis activation.

FIG. 4C shows a prediction of synchronized lysis circuit dynamics in a dual strain population using various communication motifs. Model-generated heat maps depicting time-averaged population ratio of light grey lysing strain and black lysing strain (with one strain having a strong response to the other's QS signal) in a well-mixed, constant flow co-culture, as function of light grey's growth rate α₁ against black's growth rate α₂. On the left of each heat-map is the communication motif it exhibits and experimental candidate QS systems to achieve the desired signaling characteristic. These traits determine the behavior and composition of the co-culture. The white dot on the heat map indicates the growth rate parameters selected for the time-series plots. Time series plots show population of the two strains as a function of time. Light grey dashed lines indicate the region where both strains are in the oscillatory regime, black dashed lines mark the area in which strains are self-limiting.

FIG. 4D shows a prediction of synchronized lysis circuit dynamics in a dual strain population using various communication motifs. Model-generated heat maps depicting time-averaged population ratio of light grey lysing strain and black lysing strain (with one strain having a weak response to the other's QS signal) in a well-mixed, constant flow co-culture, as function of light grey's growth rate α1 against black's growth rate α2. On the left of each heat-map is the communication motif it exhibits and experimental candidate QS systems to achieve the desired signaling characteristic. These traits determine the behavior and composition of the co-culture. The white dot on the heat map indicates the growth rate parameters selected for the time-series plots. Time series plots show population of the two strains as a function of time.

FIG. 4E shows a prediction of synchronized lysis circuit dynamics in a dual strain population using various communication motifs. Model-generated heat maps depicting time-averaged population ratio of light grey lysing strain and black lysing strain (both strains are completely orthogonal lysing strains) in a well-mixed, constant flow co-culture, as function of light grey's growth rate α1 against black's growth rate α2. On the left of each heat-map is the communication motif it exhibits and experimental candidate QS systems to achieve the desired signaling characteristic. These traits determine the behavior and composition of the co-culture. The white dot on the heat map indicates the growth rate parameters selected for the time-series plots. Time series plots show population of the two strains as a function of time. The rpa and lux systems could be used for this dynamic as they are signal orthogonal.

FIG. 4F shows a prediction of synchronized lysis circuit dynamics in a dual strain population using various communication motifs. Model-generated heat maps depicting time-averaged population ratio of light grey lysing strain and black lysing strain (two strains are completely orthogonal strains: the “light grey” strain in the weak lysis regime (leading to constant lysis), and the “black” strain in the lysis regime.) in a well-mixed, constant flow co-culture, as function of light grey's growth rate α1 against black's growth rate α2. On the left of each heat-map is the communication motif it exhibits and experimental candidate QS systems to achieve the desired signaling characteristic. These traits determine the behavior and composition of the co-culture. The white dot on the heat map indicates the growth rate parameters selected for the time-series plots. Time series plots show population of the two strains as a function of time. Two completely orthogonal strains with This is the regime corresponding to the experimental system. Oscillations in the “light grey” strain's population are imposed by the oscillatory “black” strain through volume exclusion.

FIG. 5 shows plasmid maps of the main DNA constructs used herein. “1” arrows represent LuxR. “4” arrows represent LuxI, LuxI+LAA, or LuxI+TS+LAA. “2” elements represent the pLuxI promoter. “7” arrows represent RpaR. “3” arrows represent sfGFP. “8” arrows represent CFP. “9” arrows represent RpaI. “10” arrows represent the lysis gene E. “6” elements represent antibiotic resistance markers. “5” elements represent origins of replication. Gray elements represent transcription terminators.

FIG. 6 is a graph showing dynamics of the model equation 1 in Example 1. n represents the population of cells, and q represents the amount of fluorescent protein in the system.

FIG. 7A is a schematic showing some of the QS communication possibilities between two members of a microbial consortia, where each strain is capable of either sending, receiving, both or neither, there are generally 16 possible communication motifs (FIG. 8A).

FIG. 7B is a schematic showing the QS communication possibilities between two members of a microbial consortia, where the dual-strain consortia interfaced with the synchronized lysis circuit, and show one-way orthogonal signaling

FIG. 7C is a schematic showing the QS communication possibilities between two members of a microbial consortia, where the dual-strain consortia interfaced with the synchronized lysis circuit, and show two-way orthogonal signaling.

FIG. 7D is a schematic showing how QS systems can be tested and characterized for easy categorization. A strain containing one QS promoter and one QS receptor is subjected to a range of signals and its dose response curve is quantified by the area under its curve (AUC), which becomes the heat map parameter in FIGS. 7E-G. Signaling homoserine lactones (HSL), 3-oxo-C6 HSL (3OC6), p-Coumaroyl HSL (pC), 3-oxo-C12 HSL (3OC12), and 3-oxo-C8 HSL (3OC8), are represented as pentagons color-coded to their native QS system. Error bars indicate the standard error of the mean (n=3).

FIG. 7E is a heat map of aggregated QS systems and their AUC responses to different signals; data is representative of 3 technical replicates. Square matrices with significant induction in all squares indicates two-way signal cross-talk.

This methodology allows for quick identification of signal orthogonal strains classified by their diagonal matrices (G).

FIG. 7F is a heat map of aggregated QS systems and their AUC responses to different signals; data is representative of 3 technical replicates. One-way signal cross-talk indicates only one square not on the diagonal with a significant value

FIG. 7G is a heat map of aggregated QS systems and their AUC responses to different signals; data is representative of 3 technical replicates. This methodology allows for quick identification of signal orthogonal strains classified by their diagonal matrices.

FIG. 8A is a schematic showing possible Two-Strain Quorum Sensing Communication Motifs. Each strain is capable of no, intra, inter or both intra and inter-communication. This gives 16 general QS dynamics between two strains. Certain communication motifs require different level of signal orthogonality. “#” means that motif requires complete signal orthogonality. “*” means that motif requires at least one-way signal orthogonality. Black dot means the motif doesn't necessarily need signal orthogonality, but it could utilize that dynamic.

FIG. 8B is an exemplary heat map of aggregated QS systems and their AUC responses to different signals. The meaning of the column and row label pictograms is the same as in FIG. 7.

FIG. 9A shows a fluorescence intensity heat map of individual traps plotted against time and raw CFP fluorescence time-series of non-lysis Lux-CFP cells grown alone.

FIG. 9B shows a fluorescence intensity heat map of individual traps plotted against time and raw GFP fluorescence time-series of non-lysis Rpa-GFP cells grown alone.

FIG. 9C shows a fluorescence intensity heat map of individual traps plotted against time and raw CFP fluorescence time-series of oscillatory lysing Lux-CFP cells grown alone.

FIG. 9D shows a fluorescence intensity heat map of individual traps plotted against time and raw GFP fluorescence time-series of constantly lysing Rpa-GFP cells grown alone.

### DETAILED DESCRIPTION

Microbial ecologists are increasingly turning to small, synthesized ecosystems¹⁻⁵ as a reductionist tool to probe the complexity of native microbiomes^{6,7}. Concurrently, synthetic biologists have gone from single-cell gene circuits⁸⁻¹¹ to controlling whole populations using intercellular signaling¹²⁻¹⁶. The intersection of these fields is giving rise to new approaches in waste recycling¹⁷, industrial fermentation¹⁸, bioremediation¹⁹, and human health^{16,20}. These applications share a common challenge⁷ well known in classical ecology^{21,22}; stability of an ecosystem cannot arise without mechanisms that prohibit the faster growing species from eliminating the slower. Here, orthogonal quorum sensing systems and a population control circuit with diverse self-limiting growth dynamics are combined in order to engineer two ‘ortholysis’ circuits capable of maintaining a stable co-culture of metabolically competitive strains in microfluidic devices. While no successful co-cultures are observed in a two-strain ecology without synthetic population control, the ‘ortholysis’ design dramatically increases the co-culture rate from 0% to approximately 80%. Agent-based and deterministic modeling reveal that the system can be adjusted to yield different dynamics, including phase-shifted, anti-phase or synchronized oscillations as well as stable steady-state population densities. The ‘ortholysis’ approach establishes a paradigm for constructing synthetic ecologies by developing stable communities of competitive microbes without the need for engineered codependency.

As used herein the term “co-culture” or “co-culturing” refers to growing or culturing two or more distinct cell types (e.g., at least two distinct bacterial strains) within a single recipient (e.g., a single cell culture vessel, a single cell culture plate, a single bioreactor, a single microfluidic device). Under optimal conditions of co-culturing, each of the at least two bacterial strains (e.g., each of at least a first bacterial strain and a second bacterial strain) has a positive growth rate.

A variety of different methods known in the art can be used to introduce any of the plasmids disclosed herein into a bacterial cell (e.g., a Gram negative bacterial cell, a Gram positive bacterial cell). Non-limiting examples of methods for introducing nucleic acid into a cell include: transformation, microinjection, electroporation, cell squeezing, sonoporation. Skilled practitioners will appreciate that the plasmids described herein can be introduced into any cell provided herein.

The term “treat(ment),” is used herein to denote delaying the onset of, inhibiting, alleviating the effects of, or prolonging the life of a subject suffering from disease, e.g., a cancer, an infection.

The terms “effective amount” and “amount effective to treat” as used herein, refer to an amount or concentration of a composition or treatment described herein, at least two bacterial strains (e.g., at least a first bacterial strain and a second bacterial strain), utilized for a period of time (including acute or chronic administration and periodic or continuous administration) that is effective within the context of its administration for causing an intended effect or physiological outcome. For example, effective amounts of at least two bacterial strains (e.g., at least a first bacterial strain and a second bacterial strain) that express and/or secrete a therapeutic agent (e.g., any of the therapeutic agents described herein) for use in the present disclosure include, for example, amounts that inhibit the growth of a cancer, e.g., tumor cells and/or tumor-associated immune cells, improve or delay tumor growth, improve survival for a subject suffering from or at risk of developing cancer, and improving the outcome of other cancer treatments. As another example, effective amounts of at least two bacterial strains (e.g., at least a first bacterial strain and a second bacterial strain) that express and/or secrete a therapeutic agent (e.g., any of the therapeutic agents described herein) can include amounts that advantageously affect a tumor microenvironment.

The term “subject” is used throughout the specification to describe an animal, human or non-human, to whom treatment according to the methods of the present disclosure is provided. Veterinary applications are clearly anticipated by the present disclosure. The term includes but is not limited to birds, reptiles, amphibians, and mammals, e.g., humans, other primates, pigs, rodents, such as mice and rats, rabbits, guinea pigs, hamsters, horses, cows, cats, dogs, sheep and goats. Preferred subjects are humans, farm animals, and domestic pets such as cats and dogs. In some embodiments, the subject is a human. For example, in any of the methods described herein, the subject can be at least 2 years or older (e.g., 4 years or older, 6 years or older, 10 years or older, 13 years or older, 16 years or older, 18 years or older, 21 years or older, 25 years or older, 30 years or older, 35 years or older, 40 years or older, 45 years or older, 50 years or older, 60 years or older, 65 years or older, 70 years or older, 75 years or older, 80 years or older, 85 years or older, 90 years or older, or 2, 3, 4, 5, 6, 7, 8, 9, 10, 12, 13, 14, 15, 16, 18, 20, 21, 24, 25, 27, 28, 30, 33, 35, 37, 39, 40, 42, 44, 45, 48, 50, 52, 55, 60, 65, 70, 75, 80, 85, 90, 95, 96, 97, 98, 99, 100, 101, 102, 103, or 104 years old).

The term “population” when used before a noun means two more of the specific noun. For example, the phrase “a population of bacterial strains” means two or more bacterial strains.

The term “cancer” refers to cells having the capacity for autonomous growth. Examples of such cells includes cells having an abnormal state or condition characterized by rapidly proliferating cell growth. The term is meant to include cancerous growths, e.g., tumors, oncogenic processes, metastatic tissues, malignantly transformed cells.

A metastatic tumor can arise from a multitude of primary tumor types, including but not limited to those of prostate, colon, lung, breast, bone and liver origin. Metastases develop, e.g., when tumor cells shed, detach or migrate from a primary tumor, enter the vascular system, penetrate into surrounding tissues, and grow to form tumors at distinct anatomical sites, e.g., sites separate from a primary tumor.

Individuals considered at risk for developing cancer may benefit from the present disclosure, e.g., because prophylactic treatment can begin before there is any evidence and/or diagnosis of the disorder. Individuals “at risk” include, e.g., individuals exposed to carcinogens, e.g., by consumption (e.g., by inhalation and/or ingestion), at levels that have been shown statistically to promote cancer in susceptible individuals. Also included are individuals at risk due to exposure to ultraviolet radiation, or their environment, occupation, and/or heredity, as well as those who show signs of a precancerous condition such as polyps. Similarly, individuals in very early stages of cancer or development of metastases (i.e., only one or a few aberrant cells are present in the individual's body or at a particular site in an individual's tissue) may benefit from such prophylactic treatment.

Skilled practitioners will appreciate that a patient can be diagnosed, e.g., by a medical professional, e.g., a physician or nurse (or veterinarian, as appropriate for the patient being diagnosed), as suffering from or at risk for a condition described herein, e.g., cancer, using any method known in the art, e.g., by assessing a patient's medical history, performing diagnostic tests, and/or by employing imaging techniques.

Skilled practitioners will also appreciate that treatment need not be administered to a patient by the same individual who diagnosed the patient (or the same individual who prescribed the treatment for the patient). Treatment can be administered (and/or administration can be supervised), e.g., by the diagnosing and/or prescribing individual, and/or any other individual, including the patient her/himself (e.g., where the patient is capable of self-administration).

Provided herein are methods of maintaining a co-culture by quorum sensing. In some embodiments, the methods can include co-culturing at least a first bacterial strain and a second bacterial strain during a period of time of at least 12 hours; wherein at least one of the first and second bacterial strains has a growth advantage compared to at least one other bacterial strain. In some embodiments, each of the first and second bacterial strains comprises a lysis plasmid having a lysis gene under the control of an activatable promoter; and an activator plasmid having an activator gene, the expression of which promotes the accumulation of a quorum sensing molecule, wherein both the activatable promoter of the lysis gene and the expression of the activator gene is activated by the quorum sensing molecule, wherein the quorum-sensing molecule of the first strain is different from the quorum-sensing molecule of the second strain, and wherein each quorum-sensing molecule of the first and second strains has no or substantially no effect on the activatable promoter of the lysis gene of the other strain.

As used herein, “substantially no effect” means no measurable effect on the activatable promoter, as measured by the expression of the activatable promotor of a fluorescent protein.

In some embodiments, the methods can include co-culturing multiple co-cultures such that the method can include, e.g., co-culturing, along with the first and second strains, a third bacterial strain and a fourth bacterial strain that can be described similarly to the first and second bacterial strains. In some embodiments, the co-culturing can include co-culturing one or more sets of two bacterial strains described similarly to the first and second bacterial strains, such that a first set includes the first and second strain, a second set includes a third and fourth strain, and so on. In some embodiments, each set can comprise a co-lysis (e.g., orthogonal co-lysis) circuit.

In some aspects, the lysis plasmid and activator plasmid of at least one of the first and second strains can be the same plasmid. In some aspects, the lysis plasmid and activator plasmid of at least one of the first and second strains can be separate plasmids.

In some aspects, the at least the first and second strains can be metabolically competitive.

In some aspects, the at least the first and second strains can be selected from E. coli, S. typhimurium, or a bacterial variant thereof.

In some aspects, the first strain does not have a growth advantage compared to at least one other bacterial strain. In some embodiments, the first strain does not have a growth advantage compared to at least the second bacterial strain. In some embodiments, the first strain does not have a growth advantage compared to at least one other bacterial strain in the co-culture that is not the second strain.

In one aspect, in each of the first and second strains the lysis plasmid comprises a lysis gene, an activatable promoter, and optionally a reporter gene; and the activator plasmid comprises an activator gene, a degradation tag, and optionally a reporter gene. In some embodiments, at least one reporter gene is selected from a gene encoding a green fluorescent protein (GFP), cyan fluorescent protein (CFP), red fluorescent protein (RFP), or a variant thereof. In some embodiments, the degradation tag can be an ssrA-LAA degradation tag.

In some aspects, the lysis gene in at least one of the first and second strains can be E from a bacteriophage ΦX174.

In some aspects, in the first strain the activatable promoter is a LuxR-AHL activatable luxI promoter and the activator gene is a LuxI.

In some aspects, in the second strain the activatable promoter is a RpaR-AHL activatable RpaI promoter and the activator gene is a RpaI.

In some aspects, the co-culture is inoculated at a ratio of 1:100 of the bacterial strain having the growth advantage compared to the other bacterial strain to the other bacterial strain.

In some aspects, at least one of the plasmids is integrated into a genome of at least one of the first and second strains.

In some aspects, at least one of the plasmids can further comprises a plasmid-stabilizing element. In some embodiments, the plasmid-stabilizing element is a toxin/antitoxin system or an actin-like protein partitioning system.

In some aspects, the culturing can occur in a microfluidic device.

In some embodiments, the period of time can be from about 12 to about 72 hours. In some embodiments, the period of time is selected from at least 24 hours, at least 48 hours, at least 72 hours, and at least 96 hours. In some embodiments, the period of time is selected from 12 hours, 24 hours, 48 hours, 72 hours, and 96 hours.

In some aspects, the co-culturing of the first and second strains is in a constant lysis state; wherein the constant lysis state is characterized by a steady-state balance of growth and lysis of the at least two bacterial strains.

In some aspects, the co-culturing of the at least two bacterial strains is oscillatory; wherein the oscillatory co-culturing indicates a high level of activator degradation in at least one of the two bacterial strains.

### Bacterial Strains

Also provided herein are methods of generating a recombinant bacterial cell that can express and/or secrete a therapeutic agent (e.g., any of the therapeutic agents described herein) that include: introducing into a bacterial cell a lysis plasmid, an activator plasmid, a nucleic acid encoding the therapeutic agent to be produced in the recombinant bacterial cell, and a plasmid-stabilizing element; and culturing the recombinant bacterial cell under conditions sufficient for the expression and/or secretion of the toxin, antitoxin and therapeutic agent. In some embodiments, the introducing step can include introducing into a recombinant bacterial cell an expression vector including a nucleic acid encoding the therapeutic agent to be produced into a recombinant bacterial cell. In some embodiments, the bacterial cell is an E. coli cell, a S. typhimurium cell, or a bacterial variant thereof. In some embodiments, the bacterial strain is a Gram-negative bacterial strains, e.g., a Salmonella strain, an Acetobacter strain, an Enterobacter strain, a Fusobacterium strain, a Helicobacter strain, a Klebsiella strain, or an E. coli strain. In some embodiments, the bacterial strain is a Gram-positive bacterial strain, e.g., an Actinomyces strain, a Bacillus strain, a Clostridium strain, an Enterococcus strain, or a Lactobacillus strain. In some embodiments, the at least two bacterial strains (e.g., at least a first bacterial strain and a second bacterial strain) are all Gram negative bacterial strains or all Gram positive strains. In some embodiments, at least one of the at least two bacterial strains (e.g., at least one of a first bacterial strain and a second bacterial strain) is a Gram negative bacterial strain. In some embodiments, at least one of the at least two bacterial strains (e.g., at least one of a first bacterial strain and a second bacterial strain) is a Gram positive bacterial strain.

Methods of culturing bacterial cells are well known in the art, and examples of such methods are provided in the Examples. Bacterial cells can be maintained in vitro under conditions that favor proliferation and growth. Briefly, bacterial cells can be cultured by contacting a bacterial cell (e.g., any bacterial cell described herein) with a cell culture medium that includes the necessary growth factors and supplements to support cell viability and growth.

Methods of introducing nucleic acids and expression vectors into a bacterial cell are known in the art. For example, transformation can be used to introduce a nucleic acid into a bacterial cell.

Provided herein are bacterial strain that include a lysis plasmid, a plasmid-stabilizing element, and an activator plasmid; wherein the lysis plasmid comprises a lysis gene, an activatable promoter, a therapeutic agent, and optionally a reporter gene; and the activator plasmid comprises an activator gene, a degradation tag, and optionally a reporter gene.

In some embodiments of any of the bacterial strains described herein, the lysis gene is E from a bacteriophage ΦX174.

In some embodiments of any of the bacterial strains described herein, the activatable promoter is a LuxR-AHL activatable luxI promoter and the activator gene is a LuxI.

In some embodiments of any of the bacterial strains described herein, the activatable promoter is a RpaR-AHL activatable RpaI promoter and the activator gene is a RpaI.

In some embodiments, the plasmid-stabilizing element is a toxin/antitoxin system or an actin-like protein partitioning system. In some embodiments, the plasmid-stabilizing element is a toxin/antitoxin system (e.g., type I toxin/antitoxin system, type II toxin/antitoxin system, type II toxin/antitoxin system, type IV toxin/antitoxin system, type V toxin/antitoxin system, or type VI toxin/antitoxin system). Non-limiting examples of type I toxin/antitoxins include Hok and Sok, Fst and RNAII, TisB and IstR, LdrB and RdID, FlmA and FlmB, Ibs and Sib, TxpA/BrnT and RatA, SymE and SymR, and XXCV2162 and ptaRNA1. Non-limiting examples of type II toxin/antitoxins include CcdB and CcdA; ParE and ParD; MaxF and MazE; yafO and yafN; HicA and HicB; Kid and Kis; Zeta and Epsilon; DarT and DarG. For example, type III toxin/antitoxin systems include interactions between a toxic protein and an RNA antitoxin, e.g., ToxN and ToxI. For example, type IV toxin/antitoxin systems include toxin/antitoxin systems that counteract the activity of the toxin and the two proteins do not directly interact. An example of a type V toxin/antitoxin system is GoT and GoS. An example of a type VI toxin/antitoxin system is SocA and SocB.

In some embodiments, the plasmid-stabilizing element is a bacteriocin.

Bacteriocins are ribosomally-synthesized peptides that are produced by bacteria. Bacteriocins are non-toxic to bacteria that produce the bacteriocins and are toxic to other bacteria. Most bacteriocins are extremely potent, and exhibit antimicrobial activity at nanomolecular concentrations. By way of example, eukaryotic produced microbials have 10² to 10³ lower activities (Kaur and Kaur (2015) Front. Pharmacol. doi: 10.3389).

Non-limiting examples of bacteriocins that can be included in any of the bacteria strains, systems and methods described herein include: acidocin, actagardine, agrocin, alveicin, aureocin, aureocin A53, aureocin A70, bisin, carnocin, carnocyclin, caseicin, cerein, circularin A, colicin, curvaticin, divercin, duramycin, enterocin, enterolysin, epidermin/gallidermin, erwiniocin, gardimycin, gassericin A, glycinecin, halocin, klebicin, lactosin S, lactococcin, lacticin, leucoccin, lysostaphin, macedocin, mersacidin, mesentericin, microbisporicin, microcin S, mutacin, nisin, paenibacillin, planosporicin, pediocin, pentocin, plantaricin, pneumocyclicin, pyocin, reutericin 6, sakacin, salivaricin, sublancin, subtilin, sulfolobicin, tasmancin, thuricin 17, trifolitoxin, variacin, vibriocin, warnericin, cytolisin, pyocyn S2, colicin A, colicin E1, microcin MccE492, and warnerin.

In some embodiments, the bacteriocin is obtained from a Gram negative bacteria (e.g., microcins (e.g., microcin V of E coli, subtilosin A from B. subtillis), colicins (E.g., colicin produced by and toxic to certain strains of E. coli (e.g., colicin A, colicin B, colicin E1, colicin E3, colicin E5, and colicin E7), tailocins (e.g., R-type pyocins, F-type pyocins)).

In some embodiments, the bacteriocin is obtained from a Gram positive bacteria (e.g., class I bacteriocins (e.g., Nisin, lantibiotics), class II bacteriocins (e.g., IIa pediocin-like bacteriocins, IIb bacteriocins (e.g., lactococcin G), IIc cyclic peptides (e.g., enterocin AS-48), IId single peptide bacteriocins (e.g., aureocin A53), class III bacteriocins (e.g., IIIa (e.g., bacteriolysins), and IIIb (which kill the target by disrupting the membrane potential), or class IV bacteriocins (e.g., complex bacteriocins containing lipid or carbohydrate moieties)),

In some embodiments of any of the bacterial strains described herein, the therapeutic agent is selected from the group consisting of: an inhibitory nucleic acid, a cytokine, a fusion protein, and an antibody or antigen-binding fragment thereof.

Also provided herein are methods of co-culturing at least two (e.g., at least three, at least four, at least five, at least six, at least seven, at least eight, at least nine, at least ten, at least eleven, at least twelve, 2, 3, 4, 5, 6, 7, 8, 9, 10, 11 or 12) bacterial strains (e.g., any of the bacterial strains described herein). In some embodiments, a ratio of inoculation of at least one bacterial strain to at least one other bacterial strain is between 100 000:1 and 1:100 000 (e.g., 100 000:1, 95 000:1, 90 000:1, 85 000:1, 80 000:1, 75 000:1, 70 000:1, 65 000:1, 60 000:1, 55 000:1, 50 000:1, 45 000:1, 40 000:1, 35 000:1, 30 000:1, 25 000:1, 20 000:1, 15 000:1, 10 000:1, 9000:1, 8500:1, 8000:1, 7500:1, 7000:1, 6500:1, 6000:1, 5500:1, 5000:1, 4500:1, 4000:1, 3500:1, 3000:1, 2500:1, 2000:1, 1500:1, 1000:1, 950:1, 900:1, 850:1, 800:1, 750:1, 700:1, 650:1, 600:1, 550:1, 500:1, 450:1, 400:1, 350:1, 300:1, 250:1, 200:1, 150:1, 100:1, 90:1, 80:1, 70:1, 60:1, 50:1, 40:1, 30:1, 25:1, 20:1, 18:1, 16:1, 15:1, 14:1, 12:1, 10:1, 8:1, 6:1, 4:1, 3:1, 2:1, 1:1, 1:2, 1:3, 1:4, 1:6, 1:8, 1:10, 1:12, 1:14, 1:15, 1:16, 1:18, 1:20, 1:25, 1:30, 1:40, 1:50, 1:60, 1:70, 1:80, 1:90, 1:100, 1:150, 1:200, 1:250, 1:300, 1:350, 1:400, 1:450, 1:500, 1:550, 1:600, 1:650, 1:700, 1:750, 1:800, 1:850, 1:900, 1:950, 1:1000, 1:1500, 1:2000, 1:2500, 1:3000, 1:3500, 1:4000, 1:4500, 1:5000, 1:5500, 1:6000, 1:6500, 1:7000, 1:7500, 1:8000, 1:8500, 1:9000, 1:9500, 1:10 000, 1:15 000, 1:20 000, 1:25 000, 1:30 000, 1:35 000, 1:40 000, 1:45 000, 1:50 000, 1:55 000, 1:60 000, 1:65 000, 1:70 000, 1:75 000, 1:80 000, 1:85 000, 1:90 000, 1:95 0000, 1:100 000).

In some embodiments, a ratio of inoculation of the first bacterial strain to the second bacterial strain is between 100 000:1 and 1:100 000 (e.g., 100 000:1, 95 000:1, 90 000:1, 85 000:1, 80 000:1, 75 000:1, 70 000:1, 65 000:1, 60 000:1, 55 000:1, 50 000:1, 45 000:1, 40 000:1, 35 000:1, 30 000:1, 25 000:1, 20 000:1, 15 000:1, 10 000:1, 9000:1, 8500:1, 8000:1, 7500:1, 7000:1, 6500:1, 6000:1, 5500:1, 5000:1, 4500:1, 4000:1, 3500:1, 3000:1, 2500:1, 2000:1, 1500:1, 1000:1, 950:1, 900:1, 850:1, 800:1, 750:1, 700:1, 650:1, 600:1, 550:1, 500:1, 450:1, 400:1, 350:1, 300:1, 250:1, 200:1, 150:1, 100:1, 90:1, 80:1, 70:1, 60:1, 50:1, 40:1, 30:1, 25:1, 20:1, 18:1, 16:1, 15:1, 14:1, 12:1, 10:1, 8:1, 6:1, 4:1, 3:1, 2:1, 1:1, 1:2, 1:3, 1:4, 1:6, 1:8, 1:10, 1:12, 1:14, 1:15, 1:16, 1:18, 1:20, 1:25, 1:30, 1:40, 1:50, 1:60, 1:70, 1:80, 1:90, 1:100, 1:150, 1:200, 1:250, 1:300, 1:350, 1:400, 1:450, 1:500, 1:550, 1:600, 1:650, 1:700, 1:750, 1:800, 1:850, 1:900, 1:950, 1:1000, 1:1500, 1:2000, 1:2500, 1:3000, 1:3500, 1:4000, 1:4500, 1:5000, 1:5500, 1:6000, 1:6500, 1:7000, 1:7500, 1:8000, 1:8500, 1:9000, 1:9500, 1:10 000, 1:15 000, 1:20 000, 1:25 000, 1:30 000, 1:35 000, 1:40 000, 1:45 000, 1:50 000, 1:55 000, 1:60 000, 1:65 000, 1:70 000, 1:75 000, 1:80 000, 1:85 000, 1:90 000, 1:95 0000, 1:100 000).

In some embodiments, a cycle of lysis of any of the bacterial strains described herein can be between 1 hour to 35 days (e.g., 1 hour to 30 days, 1 hour to 28 days, 1 hour to 26 days, 1 hour to 25 days, 1 hour to 24 days, 1 hour to 22 days, 1 hour to 20 days, 1 hour to 18 days, 1 hour to 16 days, 1 hour to 14 days, 1 hour to 12 days, 1 hour to 10 days, 1 hour to 8 days, 1 hour to 7 days, 1 hour to 6 days, 1 hour to 5 days, 1 hour to 4 days, 1 hour to 72 hours, 1 hour to 70 hours, 1 hour to 68 hours, 1 hour to 66 hours 1 hour to 64 hours, 1 hour to 62 hours, 1 hour to 60 hours, 1 hour to 58 hours, 1 hour to 56 hours, 1 hour to 54 hours, 1 hour to 52 hours, 1 hour to 50 hours, 1 hour to 48 hours, 1 hour to 46 hours, 1 hour to 44 hours, 1 hour to 40 hours, 1 hour to 38 hours, 1 hour to 36 hours, 1 hour to 34 hours, 1 hour to 32 hours, 1 hour to 30 hours, 1 hour to 28 hours, 1 hour to 26 hours, 1 hour to 24 hours, 1 hour to 22 hours, 1 hour to 20 hours, 1 hour to 18 hours, 1 hour to 16 hours, 1 hour to 14 hours, 1 hour to 12 hours, 1 hour to 10 hours, 1 hour to 8 hours, 1 hour to 6 hours, 1 hour to 4 hours, 1 hour to 2 hours, 2 hours to 35 days, 2 hours to 30 days, 2 hours to 28 days, 2 hours to 26 days, 2 hours to 25 days, 2 hours to 24 days, 2 hours to 22 days, 2 hours to 20 days, 2 hours to 18 days, 2 hours to 16 days, 2 hours to 14 days, 2 hours to 12 days, 2 hours to 10 days, 2 hours to 8 days, 2 hours to 7 days, 2 hours to 6 days, 2 hours to 5 days, 2 hours to 4 days, 2 hours to 72 hours, 2 hours to 70 hours, 2 hours to 68 hours, 2 hours to 66 hours 2 hours to 64 hours, 2 hours to 62 hours, 2 hours to 60 hours, 2 hours to 58 hours, 2 hours to 56 hours, 2 hours to 54 hours, 2 hours to 52 hours, 2 hours to 50 hours, 2 hours to 48 hours, 2 hours to 46 hours, 2 hours to 44 hours, 2 hours to 40 hours, 2 hours to 38 hours, 2 hours to 36 hours, 2 hours to 34 hours, 2 hours to 32 hours, 2 hours to 30 hours, 2 hours to 28 hours, 2 hours to 26 hours, 2 hours to 24 hours, 2 hours to 22 hours, 2 hours to 20 hours, 2 hours to 18 hours, 2 hours to 16 hours, 2 hours to 14 hours, 2 hours to 12 hours, 2 hours to 10 hours, 2 hours to 8 hours, 2 hours to 6 hours, 2 hours to 4 hours, 4 hours to 35 days, 4 hours to 30 days, 4 hours to 28 days, 4 hours to 26 days, 4 hours to 25 days, 4 hours to 24 days, 4 hours to 22 days, 4 hours to 20 days, 4 hours to 18 days, 4 hours to 16 days, 4 hours to 14 days, 4 hours to 12 days, 4 hours to 10 days, 4 hours to 8 days, 4 hours to 7 days, 4 hours to 6 days, 4 hours to 5 days, 4 hours to 4 days, 4 hours to 74 hours, 4 hours to 70 hours, 4 hours to 68 hours, 4 hours to 66 hours 4 hours to 64 hours, 4 hours to 64 hours, 4 hours to 60 hours, 4 hours to 58 hours, 4 hours to 56 hours, 4 hours to 54 hours, 4 hours to 54 hours, 4 hours to 50 hours, 4 hours to 48 hours, 4 hours to 46 hours, 4 hours to 44 hours, 4 hours to 40 hours, 4 hours to 38 hours, 4 hours to 36 hours, 4 hours to 34 hours, 4 hours to 34 hours, 4 hours to 30 hours, 4 hours to 28 hours, 4 hours to 26 hours, 4 hours to 24 hours, 4 hours to 24 hours, 4 hours to 20 hours, 4 hours to 18 hours, 4 hours to 16 hours, 4 hours to 14 hours, 4 hours to 14 hours, 4 hours to 10 hours, 4 hours to 8 hours, 4 hours to 6 hours, 6 hours to 35 days, 6 hours to 30 days, 6 hours to 28 days, 6 hours to 26 days, 6 hours to 25 days, 6 hours to 24 days, 6 hours to 22 days, 6 hours to 20 days, 6 hours to 18 days, 6 hours to 16 days, 6 hours to 14 days, 6 hours to 12 days, 6 hours to 10 days, 6 hours to 8 days, 6 hours to 7 days, 6 hours to 6 days, 6 hours to 5 days, 6 hours to 4 days, 6 hours to 76 hours, 6 hours to 70 hours, 6 hours to 68 hours, 6 hours to 66 hours 6 hours to 64 hours, 6 hours to 66 hours, 6 hours to 60 hours, 6 hours to 58 hours, 6 hours to 56 hours, 6 hours to 54 hours, 6 hours to 56 hours, 6 hours to 50 hours, 6 hours to 48 hours, 6 hours to 46 hours, 6 hours to 44 hours, 6 hours to 40 hours, 6 hours to 38 hours, 6 hours to 36 hours, 6 hours to 34 hours, 6 hours to 36 hours, 6 hours to 30 hours, 6 hours to 28 hours, 6 hours to 26 hours, 6 hours to 24 hours, 6 hours to 26 hours, 6 hours to 20 hours, 6 hours to 18 hours, 6 hours to 16 hours, 6 hours to 14 hours, 6 hours to 16 hours, 6 hours to 10 hours, 6 hours to 8 hours, 12 hours to 35 days, 12 hours to 30 days, 12 hours to 28 days, 12 hours to 26 days, 12 hours to 25 days, 12 hours to 24 days, 12 hours to 22 days, 12 hours to 20 days, 12 hours to 18 days, 12 hours to 16 days, 12 hours to 14 days, 12 hours to 12 days, 12 hours to 10 days, 12 hours to 8 days, 12 hours to 7 days, 12 hours to 6 days, 12 hours to 5 days, 12 hours to 4 days, 12 hours to 72 hours, 12 hours to 70 hours, 12 hours to 68 hours, 12 hours to 66 hours 12 hours to 64 hours, 12 hours to 62 hours, 12 hours to 60 hours, 12 hours to 58 hours, 12 hours to 56 hours, 12 hours to 54 hours, 12 hours to 512 hours, 12 hours to 50 hours, 12 hours to 48 hours, 12 hours to 46 hours, 12 hours to 44 hours, 12 hours to 40 hours, 12 hours to 38 hours, 12 hours to 36 hours, 12 hours to 34 hours, 12 hours to 312 hours, 12 hours to 30 hours, 12 hours to 28 hours, 12 hours to 26 hours, 12 hours to 24 hours, 12 hours to 22 hours, 12 hours to 20 hours, 12 hours to 18 hours, 12 hours to 16 hours, 12 hours to 14 hours, 1 day to 35 days, 1 day to 30 days, 1 day to 28 days, 1 day to 26 days, 1 day to 25 days, 1 day to 24 days, 1 day to 22 days, 1 day to 20 days, 1 day to 18 days, 1 day to 16 days, 1 day to 14 days, 1 day to 12 days, 1 day to 10 days, 1 day to 8 days, 1 day to 6 days, 1 day to 5 days, 1 day to 4 days, 1 day to 3 days, 1 day to 2 days, 2 days to 35 days, 2 days to 30 days, 2 days to 28 days, 2 days to 26 days, 2 days to 25 days, 2 days to 24 days, 2 days to 22 days, 2 days to 20 days, 2 days to 18 days, 2 days to 16 days, 2 days to 15 days, 2 days to 14 days, 2 days to 12 days, 2 days to 10 days, 2 days to 8 days, 2 days to 6 days, 2 days to 4 days, 2 days to 3 days, 4 days to 35 days, 4 days to 30 days, 4 days to 28 days, 4 days to 26 days, 4 days to 25 days, 4 days to 24 days, 4 days to 22 days, 4 days to 20 days, 4 days to 18 days, 4 days to 16 days, 4 days to 15 days, 4 days to 14 days, 4 days to 12 days, 4 days to 10 days, 4 days to 8 days, 4 days to 6 days, 7 days to 35 days, 7 days to 30 days, 7 days to 28 days, 7 days to 26 days, 7 days to 25 days, 7 days to 24 days, 7 days to 22 days, 7 days to 20 days, 7 days to 18 days, 7 days to 16 days, 7 days to 15 days, 7 days to 14 days, 7 days to 12 days, 7 days to 10 days, 7 days to 8 days, 14 days to 35 days, 14 days to 30 days, 14 days to 28 days, 14 days to 26 days, 14 days to 25 days, 14 days to 24 days, 14 days to 22 days, 14 days to 20 days, 14 days to 18 days, 14 days to 16 days, 14 days to 15 days, 21 days to 35 days, 21 days to 30 days, 21 days to 28 days, 21 days to 26 days, 21 days to 25 days, 21 days to 24 days, 21 days to 22 days, 28 days to 35 days, or 28 days to 30 days; 1 hour, 2 hours, 4 hours, 6 hours, 8 hours, 10 hours, 12 hours, 14 hours, 16 hours, 18 hours, 20 hours, 22 hours, 24 hours, 2 days, 3 days, 4 days, 5 days, 6 days, 7 days, 8 days, 10 days, 12 days, 14 days, 16 days, 18 days, 20 days, 21 days, 22 days, 24 days, 25 days, 26 days, 28 days, 30 days, 32 days, 34 days or 35 days).

The length of a cycle can be regulated by using strains that lyse at different ODs. Cell lysis can also be regulated by tuning the internal circuitry of the quorum sensing components, e.g., tuning of AHL degradation, tuning lysis of protein degradation, tuning of promoters to increase or decrease expression of molecules involved in the quorum sensing circuitry.

Various methods known in the art can be used to determine whether the quorum threshold is reached. For example, the quorum threshold can be measured using traditional protein quantification methods to measure the level of AHL expression in the culture medium. The quorum threshold can also be measured using reporter proteins driven by the luxI promoter. In some embodiments, the reporter protein is a fluorescent protein, a bioluminescent luciferase reporter, a secreted blood/serum or urine reporter (e.g., secreted alkaline phosphatase, soluble peptides, Gaussian luciferase).

Various methods are known in the art to determine and/or measure cell lysis. For example, cell lysis can be determined phenotypically using microscopy by the change in intensity of transmitted light and/or absorbance at various wavelengths including 600 nm light. In some embodiments, bacterial cell lysis is synchronized. In other embodiments, bacterial cell lysis is not synchronized. Synchronized lysis can be measured via optical density at 600 nm absorbance (OD600) in a plate reader or other quantitative instruments.

### Systems

Provided herein are systems that can include a co-culture of at least two bacterial strains (e.g., at least three, at least four, at least five, at least six, at least seven, at least eight, at least nine, at least ten, at least eleven, at least twelve, 2, 3, 4, 5, 6, 7, 8, 9, 10, 11, 12), wherein the at least two bacterial strains can include a first bacterial strain having at least a portion of a first synchronized lysis circuit, wherein the first synchronized lysis circuit comprises a first lysis plasmid and a first activator plasmid and wherein the first lysis plasmid is activated by the first activator plasmid.

In some embodiments described herein, a system comprises a co-culture of at least a first bacterial strain and a second bacterial strain, wherein the first bacterial strain has at least a portion of a first synchronized lysis circuit, wherein the first synchronized lysis circuit comprises a first lysis plasmid, a first activator plasmid, and a first plasmid stabilizing element, and wherein the first lysis plasmid is activated by the first activator plasmid, and wherein the second bacterial strain has at least a portion of a second synchronized lysis circuit, wherein the second synchronized lysis circuit comprises a second lysis plasmid, a second activator plasmid, and a second plasmid stabilizing element, and wherein the second lysis plasmid is activated by the second activator plasmid, and wherein the first and second synchronized lysis circuits are orthogonal in that each has no or substantially no effect upon the other.

As used herein, “substantially no effect” means no measurable effect on the activatable promoter, as measured by the expression of the activatable promotor of a fluorescent protein.

In some aspects, the first bacterial strain can include the first lysis plasmid.

In some aspects of any of the systems described herein, the first bacterial strain can include the first activator plasmid.

In some aspects of any of the systems described herein, the at least two bacterial strains further can include a second bacterial strain.

In some aspects of any of the systems described herein, the second bacterial strain can include the first activator plasmid.

In some aspects of any of the systems described herein, each of the first bacterial strain and the second bacterial strain can include the first activator plasmid.

In some aspects of any of the systems described herein, the first lysis plasmid of the first bacterial strain operates independent of at least one other bacterial strain in the co-culture. In some embodiments, the first lysis plasmid of the first bacterial strain operates independent of at least the second bacterial strain. In some embodiments, the first lysis plasmid of the first bacterial strain operates independent of at least one bacterial strain in the system that is not the second bacterial strain.

In some aspects of any of the systems described herein, the first lysis plasmid of the first bacterial strain responds to a signal generated by at least one other bacterial strain in the co-culture. In some embodiments, the first lysis plasmid of the first bacterial strain responds to a signal generated by at least the second bacterial strain. In some embodiments, the first lysis plasmid of the first bacterial strain responds to a signal generated by at least one bacterial strain in the system that is not the second bacterial strain.

In some aspects of any of the systems described herein, the signal is a quorum sensing signal. In some aspects of any of the systems described herein, the first activator plasmid encodes a quorum sensing signal. In some aspects of any of the systems described herein, the second activator plasmid encodes a quorum sensing signal. In some embodiments, the quorum sensing signal can be a quorum sensing signaling molecule. In some embodiments, one or more of the bacterial strains respond to a quorum sensing signal. In some embodiments, the quorum sensing signals for two or more of the bacterial strains are different quorum sensing signals. In some embodiments, the quorum sensing signals for two or more of the bacterial strains are the same quorum sensing signals.

In some embodiments, the quorum sensing signaling molecule for the first and second synchronized lysis circuits are orthogonal in that each has no measurable effect upon the other.

In some aspects of any of the systems described herein, the second bacterial strain has at least a portion of a second synchronized lysis circuit, wherein the second synchronized lysis circuit comprises a second lysis plasmid and a second activator plasmid.

In some aspects of any of the systems described herein, the second bacterial strain comprises the second lysis plasmid.

In some aspects of any of the systems described herein, second bacterial strain comprises the second activator plasmid.

In some aspects of any of the systems described herein, the first bacterial strain comprises the second activator plasmid.

In some aspects of any of the systems described herein, the second lysis plasmid of the second bacterial strain operates independent of at least the first bacterial strain.

In some aspects of any of the systems described herein, the second lysis plasmid of the second bacterial strain responds to a signal generated by the first bacterial strain.

In some aspects of any of the systems described herein, at least one of the at least two bacterial strains (e.g., at least one of a first bacterial strain and a second bacterial strain) has a growth advantage compared to at least one other bacterial strain. In some embodiments, at least the first bacterial strain has a growth advantage compared to at least the second bacterial strain. In some embodiments, at least the second bacterial strain has a growth advantage compared to at least the first bacterial strain. In some embodiments, at least the first bacterial strain has a growth advantage compared to a bacterial strain present in the system that is not the second bacterial strain. In some embodiments, at least the second bacterial strain has a growth advantage compared to a bacterial strain present in the system that is not the first bacterial strain.

In some embodiments, the system can contain multiple orthogonal co-lysis circuits. For example, a system described herein could include a first co-lysis circuit comprising a first bacterial strain and a second bacterial strain as described herein, as well as a second co-lysis circuit comprising a third bacterial strain and a fourth bacterial strain. In some embodiments, the third and fourth bacterial strains each comprise a lysis plasmid having a lysis gene under the control of an activatable promoter; and an activator plasmid having an activator gene, the expression of which promotes the accumulation of a quorum sensing molecule, wherein both the activatable promoter of the lysis gene and the expression of the activator gene is activated by the quorum sensing molecule, wherein the quorum-sensing molecule of the third strain is different from the quorum-sensing molecule of the fourth strain, and wherein each quorum-sensing molecule of the third and fourth strains has no or substantially no effect on the activatable promoter of the lysis gene of the other strain. In some embodiments, the third and fourth bacterial strains can be described in the same manner that the first and second bacterial strains have been described herein. In some embodiments, a system described herein can contain 3, 4, 5, 6, 7, 8, 9, 10, or more co-lysis circuits.

In some aspects of any of the systems described herein, the first bacterial strain is competitive with at least one other bacterial strain in the co-culture. In some embodiments, the first bacterial strain is competitive with at least the second bacterial strain in the co-culture. In some embodiments, the first bacterial strain is competitive with at least one other bacterial strain in the co-culture that is not the second bacterial strain.

In some aspects of any of the systems described herein, the co-culture is stable for at least 48 hours.

In some aspects of any of the systems described herein, the at least two bacterial strains (e.g., at least a first bacterial strain and a second bacterial strain) do not comprise engineered positive or negative interactions between each other.

In some aspects of any of the systems described herein, at least one of the at least two bacterial strains (e.g., at least one of a first bacterial strain and a second bacterial strain) dynamically controls its population without exogenous input.

In some aspects of any of the systems described herein, each of at least two of the at least two bacterial strains (e.g., each of at least a first bacterial strain and a second bacterial strain) dynamically controls its own population without exogenous input.

In some aspects of any of the systems described herein, the system can further include one or more plasmid stabilizing elements. In some aspects of any of the systems described herein, the plasmid stabilizing element is selected from a toxin/antitoxin system and an actin-like protein partitioning system.

In some aspects of any of the systems described herein, the first activator plasmid encodes a degradation tagging sequence.

In some aspects of any of the systems described herein, the second activator plasmid encodes a degradation tagging sequence.

In some aspects of any of the systems described herein, the first activator plasmid encodes an N-acyl homoserine lactone.

### Methods of Treatment

Provided herein are methods of treating a disease in a subject (e.g., a cancer, an infectious disease). Exemplary methods include administering to a subject in need of treatment therapeutically effective amounts of any of the bacterial strains of described herein or any pharmaceutical composition described herein, to thereby treat the disease in the subject.

In methods described herein, administering includes administering at least two bacterial strains (e.g., at least a first bacterial strain and a second bacterial strain) to the subject.

In some embodiments of methods described herein, the at least two bacterial strains include a first bacterial strain and a second bacterial strain, wherein the first bacterial strain has at least a portion of a first synchronized lysis circuit, wherein the first synchronized lysis circuit comprises a first lysis plasmid, a first activator plasmid, and a first plasmid stabilizing element, and wherein the first lysis plasmid is activated by the first activator plasmid, wherein the second bacterial strain has at least a portion of a second synchronized lysis circuit, wherein the second synchronized lysis circuit comprises a second lysis plasmid, a second activator plasmid, and a second plasmid stabilizing element, and wherein the second lysis plasmid is activated by the second activator plasmid, and wherein the first and second synchronized lysis circuits are essentially orthogonal in that each has no or substantially no effect upon the other.

In some embodiments of any of the methods described herein, the first and the second bacterial strains are different bacterial strains that each express and/or secrete a different therapeutic agent (e.g., any of the therapeutic agents described herein).

In some embodiments of any of the methods described herein, the first and the second bacterial strain do not express or secrete a therapeutic agent (e.g., any of the therapeutic agents described herein). In some embodiments of any of the methods described herein, the first and/or the second bacterial strain produce a bacteriocin (e.g., any of the bacteriocins described herein).

In some embodiments of any of the methods described herein, the subject has a cancer or an infection.

In some embodiments wherein the subject has a cancer, the cancer can be, e.g., a primary tumor, or a metastatic tumor.

In some embodiments, the cancer is a non-T-cell-infiltrating tumor.

In some embodiments of any of the methods described herein, the cancer is selected from the group consisting of: glioblastoma, squamous cell carcinoma, breast cancer, colon cancer, hepatocellular cancer, melanoma, neuroblastoma, pancreatic cancer, and prostate cancer. Treatment of multiple cancer types at the same time is contemplated by and within the present disclosure.

In some instances, the subject having the cancer may have previously received cancer treatment (e.g., any of the cancer treatments described herein).

In some embodiments of any of the methods described herein, the subject has an infection (e.g., an infectious disease). In some embodiments of any of the methods described herein, the infection is caused by an infectious agent selected from the group consisting of: Camphylobacter jejuni, Clostridium botulinium, Escherichia coli, Listeria monocytogenes and Salmonella.

Administering may be performed, e.g., at least once (e.g., at least 2-times, at least 3-times, at least 4-times, at least 5-times, at least 6-times, at least 7-times, at least 8-times, at least 9-times, at least 10-times, at least 11-times, at least 12-times, at least 13-times, or at least 14-times) a week. Also contemplated are monthly treatments, e.g., administering at least once per month for at least 1 month (e.g., at least two, three, four, five, or six or more months, e.g., 12 or more months), and yearly treatments (e.g., administration once a year for one or more years). Administration can be via any art-known means, e.g., intravenous, subcutaneous, intraperitoneal, oral, and/or rectal administration, or any combination of known administration methods.

As used herein, treating includes “prophylactic treatment”, which means reducing the incidence of or preventing (or reducing the risk of) a sign or symptom of a disease (e.g., a cancer, an infection) in a subject at risk of developing a disease (e.g., a cancer, an infection). The term “therapeutic treatment” refers to reducing signs or symptoms of a disease, e.g., reducing cancer progression, reducing severity of a cancer, and/or re-occurrence in a subject having cancer, reducing inflammation in a subject, reducing the spread of an infection in a subject.

### Cancer

The methods described herein can be used in cancer treatments. Non-limiting examples of cancer include: acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and para-nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms' tumor.

For example, any of the methods described herein can be used to treat a cancer selected from the group consisting of: glioblastoma, squamous cell carcinoma, breast cancer, colon cancer, hepatocellular cancer, melanoma, neuroblastoma, pancreatic cancer, and prostate cancer.

### Therapeutic Agents

The term “therapeutic agent” refers to a therapeutic treatment that involves administering to a subject a therapeutic agent that is known to be useful in the treatment of a disease, e.g., a cancer, an infection. For example, a cancer therapeutic agent can decrease the size or rate of tumor growth. In other instances, a cancer therapeutic agent can affect the tumor microenvironment.

Non-limiting examples of therapeutic agents that can be expressed and/or secreted in any of the bacterial strains described herein include: an inhibitory nucleic acid (e.g., a microRNA, a short hairpin RNA, a small interfering RNA, an antisense), a cytokine, a chemokine, a toxin (e.g., a diphtheria toxin, a gelonin toxin, anthrax toxin), an antimicrobial peptide, a fusion protein, and an antibody or antigen-binding fragment thereof.

In some instances, the therapeutic agent is a therapeutic polypeptide. In some instances, the therapeutic polypeptide includes one or more polypeptides (e.g., 2, 3, 4, 5, or 6). In some instances, the therapeutic polypeptide is conjugated to a toxin, a radioisotope, or a drug via a linker (e.g., a cleavable linker, a non-cleavable linker).

In some instances, the therapeutic agent is cytotoxic or cytostatic to a target cell.

The phrase “cytotoxic to a target cell” refers to the inducement, directly or indirectly, in the death (e.g., necrosis or apoptosis) of the target cell. For example, a target cell can be a cancer cell (e.g., a cancerous cell or a tumor-associated immune cell (e.g., macrophage) or an infected cell.

The phrase “cytostatic to a target cell” refers to direct or indirect decrease in the proliferation (cell division) of a target cell in vivo or in vitro. When a therapeutic agent is cytostatic to a target cell, the therapeutic agent can, e.g., directly or indirectly result in cell cycle arrest of the target cell. In some examples, the therapeutic agent that is cytostatic can reduce the number of target cells in a population of cells that are in S phase (as compared to the number of target cells in a population of cells that are in S phase prior to contact with the therapeutic agent). In some instances, the therapeutic agent that is cytostatic can reduce the percentage of target cells in S phase by at least 20% (e.g., at least 40%, at least 60%, at least 80%) as compared to the percentage of target cells in a population of cells that in S phase prior to contact with the therapeutic agent.

### Pharmaceutical Compositions and Kits

Also provided herein are pharmaceutical compositions that include at least two (e.g., 2, 3, 4, 5, 6, 7, 8, 9, or 10) of any of the bacterial strains described herein that express and/or secrete at least one of any of the therapeutic agents described herein.

The pharmaceutical compositions can be formulated in any matter known in the art. The pharmaceutical compositions are formulated to be compatible with their intended route of administration (e.g., intravenous, subcutaneous, intraperitoneal, rectal, or oral). In some embodiments, the pharmaceutical composition is administered directly into the site of disease or diseased tissue, e.g., administered into the tumor, administered into the infected tissue. In some embodiments, administering is targeting, e.g., the pharmaceutical composition includes a targeting moiety (e.g., a targeting protein or peptide).

In some embodiments, the pharmaceutical compositions can include a pharmaceutically acceptable carrier (e.g., phosphate buffered saline). Single or multiple administrations of formulations can be given depending on for example: the dosage (i.e., number of bacterial cells per mL) and the frequency as required and tolerated by the subject. The dosage, frequency and timing required to effectively treat a subject may be influenced by the age of the subject, the general health of the subject, the severity of the disease, previous treatments, and the presence of comorbidities (e.g., diabetes, cardiovascular disease). The formulation should provide a sufficient quantity of active agent to effectively treat, prevent or ameliorate conditions, diseases or symptoms. Toxicity and therapeutic efficacy of compositions can be determined using conventional procedures in cell cultures, pre-clinical models (e.g., mice, rats, or monkeys), and humans. Data obtained from in vitro assays and pre-clinical studies can be used to formulate the appropriate dosage of any compositions described herein (e.g., pharmaceutical compositions described herein).

Efficacy of any of the compositions described herein can be determined using methods known in the art, such as by the observation of the clinical signs of a disease (e.g., tumor size, presence of metastasis).

Also provided herein are kits that include at least three of any of the bacterial strains described herein that express and/or secrete at least one of any of the therapeutic agents described herein. In some instances, the kits can include instructions for performing any of the methods described herein. In some embodiments, the kits can include at least one dose of any of the pharmaceutical compositions described herein. The kits described herein are not so limited; other variations will be apparent to one of ordinary skill in the art.

### Embodiments

1. A method of maintaining a co-culture by quorum sensing, the method comprising:
co-culturing at least a first bacterial strain and a second bacterial strain during a period of time of at least 12 hours; wherein:
at least one of the first and second bacterial strains has a growth advantage compared to at least one other bacterial strain;
each of the first and second bacterial strains comprises:
   a lysis plasmid having a lysis gene under the control of an activatable promoter; and
   an activator plasmid having an activator gene, the expression of which promotes the accumulation of a quorum sensing molecule,
   wherein both the activatable promoter of the lysis gene and the expression of the activator gene is activated by the quorum sensing molecule,
   wherein the quorum-sensing molecule of the first strain is different from the quorum-sensing molecule of the second strain, and
   wherein each quorum-sensing molecule of the first and second strains has no or substantially no effect on the activatable promoter of the lysis gene of the other strain.
      2. The method of embodiment 1, wherein the lysis plasmid and activator plasmid of at least one of the first and second strains is the same plasmid.
      3. The method of any one of embodiments 1-2, wherein the lysis plasmid and activator plasmid of at least one of the first and second strains are separate plasmids.
      4. The method of any one of embodiments 1-3, wherein the at least the first and second strains are metabolically competitive.
      5. The method of any one of embodiments 1-4, wherein the at least the first and second strains are selected from E. coli, S. typhimurium, or a bacterial variant thereof.
      6. The method of any one of embodiments 1-5, wherein the first strain does not have a growth advantage compared to the second bacterial strain.
      7. The method of any one of embodiments 1-6, wherein in each of the first and second strains the lysis plasmid comprises a lysis gene, an activatable promoter, and optionally a reporter gene; and the activator plasmid comprises an activator gene, a degradation tag, and optionally a reporter gene.
      8. The method of any one of embodiments 1-7, wherein the lysis gene in at least one of the first and second strains is E from a bacteriophage ΦX174.
      9. The method of any one of embodiments 1-8, wherein in the first strain the activatable promoter is a LuxR-AHL activatable luxI promoter and the activator gene is a LuxI.
      10. The method of any one of embodiments 1-9, wherein in the second strain the activatable promoter is a RpaR-AHL activatable RpaI promoter and the activator gene is a RpaI.
      11. The method of embodiment 7, wherein at least one reporter gene is selected from a gene encoding a green fluorescent protein (GFP), cyan fluorescent protein (CFP), red fluorescent protein (RFP), or a variant thereof.
      12. The method of embodiment 7, wherein the degradation tag is an ssrA-LAA degradation tag.
      13. The method of any one of embodiments 1-12, wherein the co-culture is inoculated at a ratio of 1:100 of the bacterial strain having the growth advantage compared to the other bacterial strain to the other bacterial strain.
      14. The method of any one of embodiments 1-13, wherein at least one of the plasmids is integrated into a genome of at least one of the first and second strains.
      15. The method of any one of embodiments 1-14, wherein at least one of the plasmids further comprises a plasmid-stabilizing element.
      16. The method of embodiment 15, wherein the plasmid-stabilizing element is a toxin/antitoxin system or an actin-like protein partitioning system.
      17. The method of any one of embodiments 1-16, wherein the culturing occurs in a microfluidic device.
      18. The method of any one of embodiments 1-17, wherein the period of time is 12 to 72 hours.
      19. The method of any one of embodiments 1-17, wherein the period of time is selected from at least 24 hours, at least 48 hours, at least 72 hours, and at least 96 hours.
      20. The method of any one of embodiments 1-17, wherein the period of time is selected from 12 hours, 24 hours, 48 hours, 72 hours, and 96 hours.
      21. The method of any one of embodiments 1-20, wherein the co-culturing of the first and second strains is in a constant lysis state; wherein the constant lysis state is characterized by a steady-state balance of growth and lysis of the at least two bacterial strains.
      22. The method of any one of embodiments 1-20, wherein the co-culturing of the at least two bacterial strains is oscillatory; wherein the oscillatory co-culturing indicates a high level of activator degradation in at least one of the two bacterial strains.
      23. A bacterial strain comprising a lysis plasmid and an activator plasmid; wherein the lysis plasmid comprises a lysis gene, an activatable promoter, and optionally a reporter gene; and the activator plasmid comprises an activator gene, a degradation tag, and optionally a reporter gene.
      24. The bacterial strain of embodiment 23, wherein the lysis gene is E from a bacteriophage ΦX174.
      25. The bacterial strain of any one of embodiments 23-24, wherein the activatable promoter is a LuxR-AHL activatable luxI promoter and the activator gene is a LuxI.
      26. The bacterial strain of any one of embodiments 23-24, wherein the activatable promoter is a RpaR-AHL activatable RpaI promoter and the activator gene is a RpaI.
      27. The bacterial strain of any one of embodiments 23-26, wherein the bacterial strain further comprises a nucleic acid encoding a therapeutic agent.
      28. The bacterial strain of embodiment 27, wherein the therapeutic agent is selected from the group consisting of: an inhibitory nucleic acid, a cytokine, a fusion protein, and an antibody or antigen-binding fragment thereof.
      29. The bacterial strain of any one of embodiments 27-28, wherein the therapeutic agent is a therapeutic polypeptide.
      30. The bacterial strain of any one of embodiments 27-29, wherein the therapeutic agent is cytotoxic or cytostatic to a target cell.
      31. The bacterial strain of any one of embodiments 27-30, wherein the target cell is a cancer cell or an infected cell.
      32. A pharmaceutical composition comprising any of the bacterial strains of embodiments 23-31.
      33. The pharmaceutical composition of embodiment 32, wherein the pharmaceutical composition is formulated for in situ drug delivery.
      34. A system comprising:
a co-culture of at least a first bacterial strain and a second bacterial strain,
wherein the first bacterial strain has at least a portion of a first synchronized lysis circuit, wherein the first synchronized lysis circuit comprises a first lysis plasmid, a first activator plasmid, and a first plasmid stabilizing element, and wherein the first lysis plasmid is activated by the first activator plasmid, and
wherein the second bacterial strain has at least a portion of a second synchronized lysis circuit, wherein the second synchronized lysis circuit comprises a second lysis plasmid, a second activator plasmid, and a second plasmid stabilizing element, and wherein the second lysis plasmid is activated by the second activator plasmid, and
wherein the first and second synchronized lysis circuits are orthogonal in that each has no or substantially no effect upon the other.
   35. The system of embodiment 34, wherein the first bacterial strain comprises the first lysis plasmid.
   36. The system of any one of embodiments 34-35, wherein the first bacterial strain comprises the first activator plasmid.
   37. The system of any one of embodiments 34-36, wherein the first plasmid stabilizing element is selected from a toxin/antitoxin system or an actin-like protein partitioning system.
   38. The system of any one of embodiments 34-37, wherein the first plasmid stabilizing element of the first bacterial strain comprises a first nucleic acid encoding a first toxin and a second nucleic acid encoding a second antitoxin.
   39. The system of any one of embodiments 34-38, wherein the second bacterial strain comprises the first activator plasmid.
   40. The system of any one of embodiments 34-39, wherein each of the first bacterial strain and the second bacterial strain comprise the first activator plasmid.
   41. The system of any one of embodiments 34-40, wherein the first lysis plasmid of the first bacterial strain operates independent of at least one other bacterial strain in the co-culture.
   42. The system of any one of embodiments 34-41, wherein the first lysis plasmid of the first bacterial strain responds to a signal generated by at least one other bacterial strain in the co-culture.
   43. The system of any one of embodiments 34-42, wherein the second bacterial strain comprises the second lysis plasmid.
   44. The system of any one of embodiments 34-43, wherein the second bacterial strain comprises the second activator plasmid.
   45. The system of any one of embodiments 34-44, wherein the first bacterial strain comprises the second activator plasmid.
   46. The system of any one of embodiment 40, wherein the signal is a quorum sensing signal.
   47. The system of any one of embodiments 34-46, wherein at least one of the first and second strains has a growth advantage compared to at least one other bacterial strain.
   48. The system of any one of embodiments 34-47, wherein the first bacterial strain is competitive with at least one other bacterial strain in the co-culture.
   49. The system of any one of embodiments 34-48, wherein the co-culture is stable for at least 48 hours.
   50. The system of any one of embodiments 34-49, wherein the first activator plasmid, the second activator plasmid, or both, encodes a degradation tagging sequence.
   51. The system of any one of embodiments 34-50, wherein the first activator plasmid encodes an N-acyl homoserine lactone.
   52. A drug delivery system comprising the system of any one of embodiments 34-51.
   53. A periodic drug delivery system comprising the system of any one of embodiments 34-51.
   54. A method of treating a disease in a subject, the method comprising:
administering to a subject in need therapeutically effective amounts of any of the bacterial strains of embodiments 23-31 or a pharmaceutical composition of embodiment of any one of embodiments 32-33, to thereby treat the disease in the subject.
   55. The method of embodiment 54, wherein administering comprises administering at least two bacterial strains to the subject.
   56. The method of any one of embodiments 54-55, wherein the at least two bacterial strains include a first bacterial strain and a second bacterial strain,
wherein the first bacterial strain has at least a portion of a first synchronized lysis circuit, wherein the first synchronized lysis circuit comprises a first lysis plasmid, a first activator plasmid, and a first plasmid stabilizing element, and wherein the first lysis plasmid is activated by the first activator plasmid,
wherein the second bacterial strain has at least a portion of a second synchronized lysis circuit, wherein the second synchronized lysis circuit comprises a second lysis plasmid, a second activator plasmid, and a second plasmid stabilizing element, and wherein the second lysis plasmid is activated by the second activator plasmid, and
wherein the first and second synchronized lysis circuits are orthogonal in that each has no or substantially no effect upon the other.
   57. The method of any one of embodiments 54-56, wherein administering comprises administering sequentially each of the at least two bacterial strains to the subject.
   58. The method of any one of embodiments 54-57, wherein administering comprises administering each of the at least two bacterial strains simultaneously.
   59. The method of any one of embodiments 54-58, wherein each of the at least two bacterial strains expresses a different therapeutic agent.
   60. The method of any one of embodiments 54-59, wherein the disease is cancer or an infection.
   61. The method of embodiment 60, wherein the infection is caused by an infectious agent selected from the group consisting of: Camphylobacter jejuni, Clostridium botulinium, Escherichia coli, Listeria monocytogenes and Salmonella.
   62. The method of embodiment 60, wherein the cancer is selected from the group consisting of: glioblastoma, squamous cell carcinoma, breast cancer, colon cancer, hepatocellular cancer, melanoma, neuroblastoma, pancreatic cancer, and prostate cancer.
   63. The method of any one of embodiments 54-62, wherein the subject has previously received treatment.
   64. The method of any one of embodiments 54-63, wherein administering occurs at least once a week.
   65. The method of any one of embodiments 54-64, wherein administering is via intravenous, subcutaneous, intraperitoneal, rectal, oral administration, or combinations thereof.
   66. The method of any one of embodiments 54-64, wherein administering is intratumoral or within the site of disease.
   67. The method of any one of embodiments 1-33, wherein the co-culturing of the first bacterial strain and the second bacterial strain occurs at a ratio of the first bacterial strain to the second bacterial strain of between about 1:100,000 and 100,000:1.
   68. The system of any one of embodiments 34-53, wherein the second lysis plasmid of the second bacterial strain operates independent of at least the first bacterial strain.
   69. The system of any one of embodiments 34-53 or 68, wherein the second lysis plasmid of the second bacterial strain responds to a signal generated by the first bacterial strain.
   70. The system of any one of embodiments 34-53 or 68-69, wherein the first activator plasmid encodes a quorum sensing signal.
   71. The system of any one of embodiments 34-53 or 68-70, wherein the second activator plasmid encodes a quorum sensing signal.
   72. The system of any one of embodiments 34-53 or 68-71, wherein the at least two bacterial strains do not comprise engineered positive or negative interactions between each other.
   73. The system of any of embodiments 34-53 or 68-72, wherein at least one of the at least two bacterial strains dynamically controls its population without exogenous input.
   74. The system of any of embodiments 34-53 or 68-73, wherein each of at least two of the at least two bacterial strains dynamically controls its own population without exogenous input.
   75. The system of any of embodiments 34-53 or 68-74, wherein the system further comprises one or more plasmid stabilizing elements.
   76. The system of embodiment 75, wherein the plasmid stabilizing element is selected from a toxin/antitoxin system and an actin-like protein partitioning system.
   77. The system of any one of embodiments 34-53 or 68-76, wherein the second activator plasmid encodes a degradation tagging sequence.
   78. A microfluidic sample trap comprising the system of any one of embodiments 34-53 or 68-77.
   79. A microfluidic device comprising one or more microfluidic sample traps of embodiment 78.
   80. The microfluidic device of embodiment 79, further comprising at least one channel in fluid communication with the microfluidic sample trap.
   81. The method of any one of embodiments 1-33, wherein the first strain and second strain are co-cultured at a ratio of from about 1:100 to about 100:1 of the first strain to the second strain.

### EXAMPLES

The invention is further described in the following examples, which do not limit the scope of the invention described in the claims.

### Example 1—Materials and Methods

### Plasmids and Strains

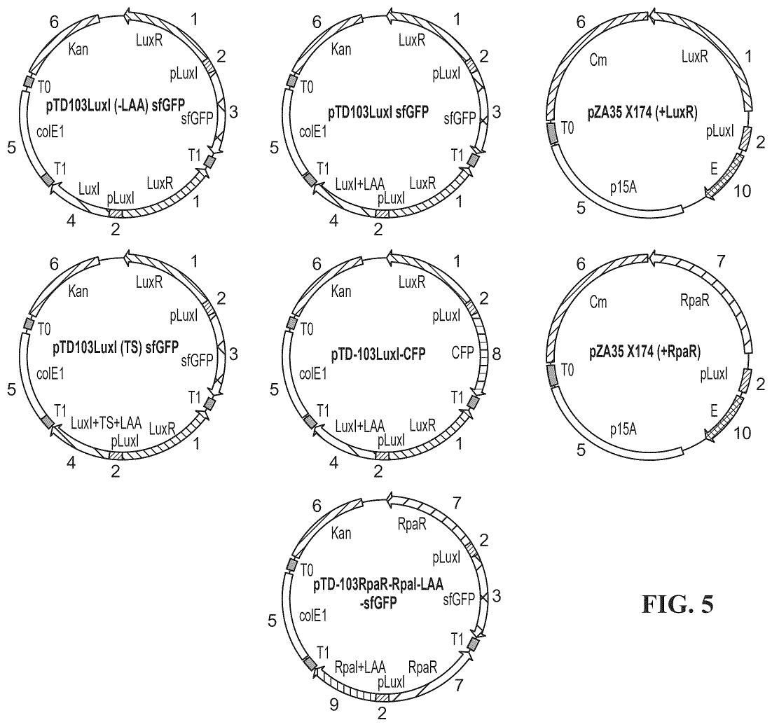

The circuit strains without the lysis plasmid were cultured in LBmedia with 50 μgml⁻¹ kanamycin, in a 37° C. incubator. The circuit strains with the lysis plasmid were cultured in the same way but with 34 μg ml⁻¹ of chloramphenicol as well along with 0.2% glucose. For microscopy and plate reader experiments 1 nM of 3-oxo-C6-HSL was added to all media. Plasmids were constructed using the CPEC method of cloning or using standard restriction digest/ligation cloning. The lux activator plasmid (Kan, ColE1) and lux-lysis plasmid (Chlor, p15A) were used in previous work^{16,31}. The RpaR and RpaI genes were obtained via PCR off the Rhodopseudomonas palustris genome obtained through ATCC to create the Rpa-activator and Rpa-lysis plasmids. The lux-sfGFP lysis circuit alone was characterized in E. coli. Co-culturing was performed with nonmotile S. typhimurium, SL1344. The SLC, in both the Lux and Rpa case, is composed of an activator plasmid and a lysis plasmid. For the circuit characterization experiments, there were three variations of the activator plasmid. The first is pTD103LuxI-sfGFP which was used in previous work³¹. This plasmid contains a LuxI with the ssrA-LAA degradation tag (amino-acid sequence of AANDENYALAA) and sfGFP, a superfolding green fluorescent protein variant³². pTD103LuxI (TS) sfGFP was constructed by adding the TS-linker (amino acid sequence of TS) between the ssrA-LAA tag and LuxI. pTD103LuxI (-LAA) sfGFP was constructed by removing the ssrA-LAA tag from LuxI. For the dual lysis experiments, the Lux-CFP strain used the activator plasmid with the ssrA-LAA tagged LuxI instead with a CFP in place of the sfGFP. The Rpa-GFP strain's activator plasmid was created by replacing LuxR with RpaR, and the LuxI with an ssrA-LAA tagged RpaI. The lysis plasmids have a p15a origin of replication and a chloramphenicol resistance marker³³ and have been previously described¹⁶. The lysis gene, E from the bacteriophage ΦX174, was kindly provided by Lingchong You and was taken from the previously reported ePop plasmid via PCR³⁴. The E gene was placed under the expression of the LuxR-AHL activatable luxI promoter for both the Lux-CFP and Rpa-GFP strains. Most of the construction was done using the CPEC method of cloning³⁵. See FIG. 5 and Table 1 for maps of the the plasmids used herein.

TABLE 1
List of strains used and their respective chassis and plasmid(s)

| Strain | Strain | Host | |
|---|---|---|---|
| # | Name | Bacterium | Plasmid(s) |
| 1 | MOD41 | JS006, | pTD103luxI(−LAA)sfGFP + |
| | | BW25113 | pZA35 X174E (+LuxR) |
| 2 | MOD29 | JS006, | pTD103luxI sfGFP + |
| | | BW25113 | pZA35 X174E (+LuxR) |
| 3 | MOD42 | JS006, | pTD103luxI(TS) sfGFP + |
| | | BW25113 | pZA35 X174E (+LuxR) |
| 4 | SRS732 | SL1344 | pTD103-LuxI-CFP |
| 5 | SRS800 | SL1344 | pTD103-LuxI-CFP + |
| | | | pZA35-X174E (+LuxR) |
| 6 | SRS840 | SL1344 | pTD103-RpaR-RPaI-LAA-sfGFP |
| 7 | SRS841 | SL1344 | pTD103-RpaR-RPaI-LAA-sfGFP + |
| | | | pZA35-X174E (+RpaR) |

JS006, BW25113 is a lab strain E. coli chassis. SL1344 is an attenuated Salmonella enterica subsp. enterica serovar Typhimurium host. The components of the plasmids listed in the table are shown in FIG. 5.

### Microfluidics and Microscopy

The microscopy and microfluidics techniques used in Example 1 have been previously described¹⁴. In short, micron-scale features are baked onto silicon wafers using cross-linked photoresist. The microfluidic device resin, PDMS (polydimethysiloxane), is then poured over the wafers and solidified by baking. The PDMS, which contains numerous devices, is peeled off and individual devices are cut out from the whole. Holes are then punched into the device at their input and output where the fluid lines will eventually plug in. After puncturing, the devices are bonded onto glass coverslips via plasma-activation. The devices were then put in a vacuum and the outlet was loaded with cells and the inlet with media. Vacuum pressure loads cells into the traps and media lines are plugged in before the cells can contaminate the upstream section of the device. The flow was then adjusted by changing the relative heights of the syringes, which for all experiments the meniscus of the media was set to one inch above the meniscus of the waste, resulting in a low, constant hydrostatic pressure driven flow.

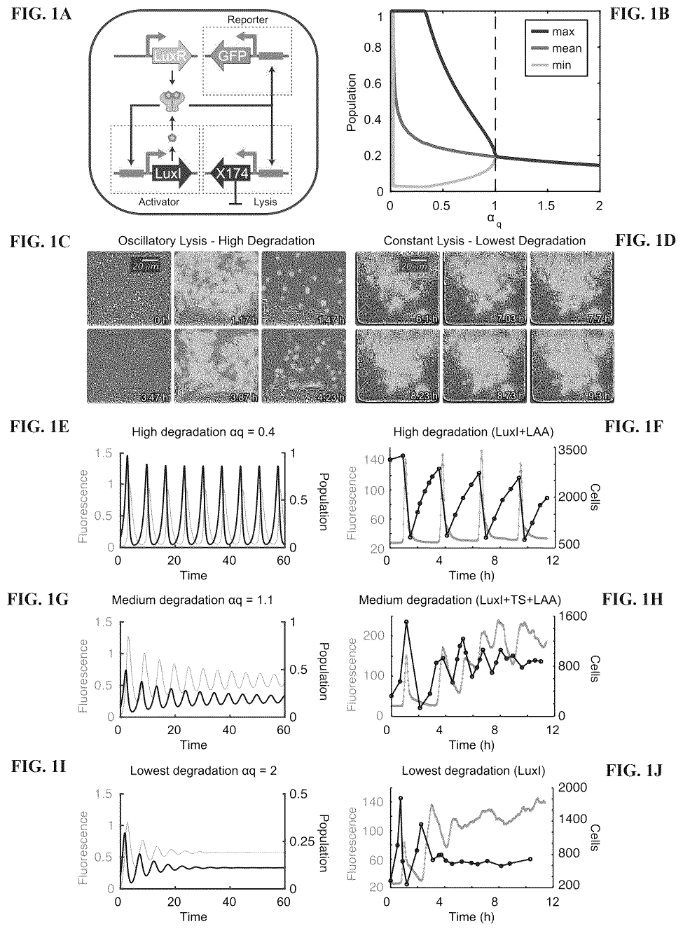

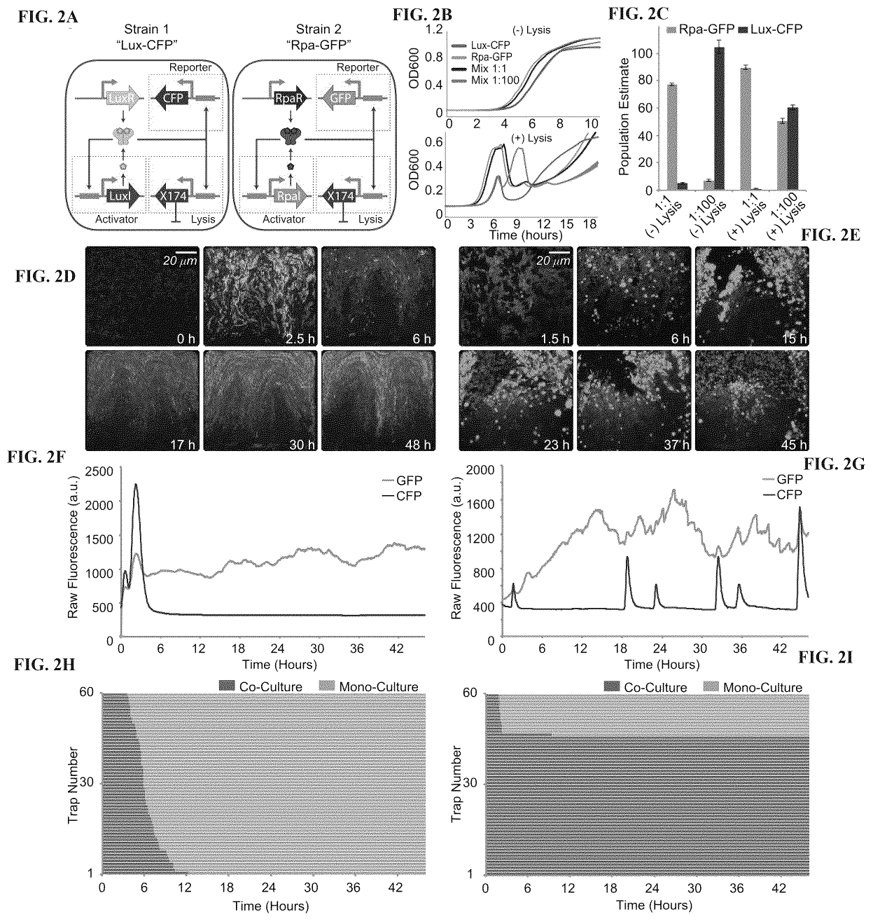

All microfluidic experiments were done in a side-trap array device as previously described¹⁴, and in all cases 0.075% Tween20 was added to the media to deter cells from sticking to the channels and the ports of the device. The bacteria growth chambers were 100 μm wide 85 μm deep and approximately 1.6 μm in height. For lysis characterization (FIG. 1): Cells were cultured until they reached an optical density of approximately 0.1 (Plastibrand 1.5 mL cuvettes were used) at which point they were spun-down and loaded via vacuum pressure the chip. Media was LB with Kanamycin and Chloramphenicol. For dual lysis and co-culturing experiments (FIG. 2): Cells were cultured until they reached an optical density of approximately 0.1 (Plastibrand 1.5 mL cuvettes were used to test OD) and 1.5 mL was spun down and resuspended in 50 μl of media. This concentrate was used to vacuum load the cells for single strain experiments, or it was mixed at a 10:1 ratio (Lux-CFP:Rpa:GFP) in the coculturing experiments before loading via vacuum pressure. Media was LB with Kanamycin (and Chloramphenicol for lysis experiments) with 1 nM 3OC6 HSL added. The microscope system used has also been previously described³¹. In short, a Nikon Eclipse TI epifluorescent microscope with phase-contrast based imaging was used. The camera is a Photometrics CoolSNAP HQ2 CCD. The acquisition software used is Nikon Elements. The microfluidic devices are housed in a plexiglass incubation chamber that is maintained at 37° C. by a heating unit. For dual lysis and co-culturing experiments: Phase-contrast images were taken at 20× magnification with 50-200 ms exposure times. Fluorescent imaging at 20× was performed at 300 ms for GFP, 30% setting on the Lumencor SOLA light source, and 300 ms and 35% for CFP. Images were taken every 3 minutes for the course of the experiment (˜2 days). Co-culture was determined to be lost if the fluorescence of either CFP or GFP went below background fluorescence, and then was checked manually in cases of the oscillatory lysing CFP strain which can go below threshold between lysis events. For the lysis characterization (FIG. 1), cells were counted using the following strategies: for experiments where the cell population was mostly aggregated together (non-sparse population), the average area of an individual bacterial cell and the average void fraction (open space between bacteria in the trap) were estimated. Taking into account the pixel density of the image, the area of the trap taken up by cells was measured using ImageJ and divided by the average area of a bacterial cell. This value was then multiplied by (1-void fraction) to yield the total estimated number of cells in the trap. Bacteria that were not close to the main group of cells were counted individually and added to the final number. For experiments where the growing population was sparse (due to the constant lysis regime), the Trainable Weka Segmentation plug-in for ImageJ was used to count cells. Plots were generated by using MATLAB. For co-culture experiments: Co-culture was determined to be lost if the fluorescence of either CFP or GFP went below background fluorescence, and then images were checked manually in cases of the oscillatory lysing CFP strain which can go below threshold between lysis events.

### Plate Reader Fluorescence and Population Estimates

For the well-plate experiments the strains were grown in a standard Falcon tissue culture 96-well flat bottom plate with appropriate antibiotics (kanamycin only for non-lysis and kanamycin and chloramphenicol for lysis strains). For consistency with microfluidic experiments, 1 nM of 3OC6-HSL was added to all media. The bacterial strains used in FIG. 2B were grown in 4 mL cultures to an optical density of 0.15 before adding 10 μL of this culture to 10 mL of fresh LB with appropriate antibiotics and added HSL. For single strain tests, 200 μl of the dilution was distributed into the well-plate. For the 1:1 mixtures, 100 μl of each dilution was added to the same well. For the 1:100 mixtures 200 μl of the Lux-CFP dilution was added with 2 μl of the Rpa-GFP dilution. For all cases there were four technical replicates. These dilutions were then grown for 10 hours (non-lysing), or 19 hours (with lysis) and their OD600 nm, GFP, and CFP levels were measured every 10 minutes in a Tecan Infinite M200 Pro. GFP readings had an excitation of 485 nm and emission of 520 nm. CFP readings had a an excitation of 433 nm and emission of 475 nm. The resulting fluorescence curves were used to calculate estimated populations of the co-cultures. Population estimates in the co-culture mixtures was estimated in the following way. The GFP fluorescence time-series trace of Rpa-GFP alone was integrated and used as a standard for accumulated fluorescence of a culture with 100% of the Rpa-GFP strain. In the same way, the CFP fluorescence time-series trace of Lux-CFP alone was integrated and used as a standard for accumulated fluorescence of a culture with 100% of the Lux-CFP strain. The integrated GFP and CFP fluorescence curves of the mixtures was then divided by the standards to give a population estimate of Rpa-GFP and Lux-CFP, respectively. For all cases, the area of the background fluorescence was substracted. Additionally, the GFP fluorescence required extra signal normalization because the Tecan's GFP sensor reads into the CFP emission profile (but not the other way around). Here are the equations used to calculate the population estimates with appropriate filtering and normalization:

$${Population}_{Lux} = \frac{Area ( {CFP}_{mix} ) - Area ( {BG}_{CFP} )}{Area ( {CFP}_{Lux} ) - Area ( {BG}_{CFP} )}$$

$$η = \frac{Area ( {GFP}_{Lux} ) - Area ( {BG}_{GFP} )}{Area ( {CFP}_{Lux} ) - Area ( {BG}_{CFP} )}$$

$${GFP}_{Crosstalk} = [ Area ( {GFP}_{mix} ) - Area ( {BG}_{GFP} ) ]$$

$${GFP}_{Real} = {GFP}_{Crosstalk} - [ Area ( {CFP}_{mix} ) - Area ( {BG}_{CFP} ) ] η$$

$${Population}_{Rpa} = \frac{{GFP}_{Real}}{Area ( {GFP}_{Rpa} ) - Area ( {BG}_{GFP} )}$$

Population_{Lux} is the population estimate of the Lux-CFP strain in a co-culture. Area(CFPₘᵢₓ) is the area of the CFP fluorescence time-series curve of a given co-culture. Area(BG_{CFP}) is the area of the background CFP fluorescence time-series line. Area(CFP_{Lux}) is the average area of the CFP fluorescence time-series curve in the wells with only the Lux-CFP strain. Area(GFP_{Lux}) is the average area of the GFP fluorescence time-series curve in the wells with only the Lux-CFP strain (For this strain the GFP fluorescence should technically be at background, further normalization is done because the Tecan's GFP sensor reads into the CFP emission profile). Area(BG_{GFP}) is the area of the background GFP fluorescence time-series line. η is the calculated fluorescence emission cross-talk scalar, and is only needed to scale GFP values as the CFP sensor does not read any GFP. The normalized, filtered, GFP value is thus given by GFP_{Real}. Area(GFPmix) is the area of the GFP fluorescence time-series curve of a given co-culture. Area(GFP_{Rpa}) is the average area of the GFP fluorescence time-series curve in the wells with only the Rpa-GFP strain. Finally, Population_{Rpa} is the population estimate of the Lux-CFP strain in a co-culture.

### Agent-Based Modeling

For the agent-based model, to simulate bacterial motion, a mechanical agent-based model was adapted from previous work^{36,37}. Each cell is modeled as a spherocylinder of unit diameter that grows linearly along its axis and divides equally after reaching a critical length l_{d}=4. It can also move along the plane due to forces and torques produced by interactions with other cells. The slightly inelastic cell-cell normal contact forces are computed via the standard spring-dashpot model, and the tangential forces are computed as velocity-dependent friction. To describe the intracellular dynamics of each cell, the ordinary differential equation model was adapted from another study¹⁶. Specifically, the intracellular dynamics are

$${P}_{lux} = {α}_{0} + {α}_{H} \frac{{( \frac{{H}_{i}}{{H}_{0}} )}^{m}}{1 + {( \frac{{H}_{i}}{{H}_{0}} )}^{m}}$$

$$\frac{{dH}_{i}}{dt} = b \frac{{I}_{i}}{{K}_{I} + {I}_{i}} + {D}_{m} ( {H}_{e} ( {x}_{i} , t ) - {H}_{i} )$$

$$\frac{{dI}_{i}}{dt} = {C}_{I} {P}_{lux} - {γ}_{I} {I}_{i}$$

$$\frac{{dL}_{i}}{dt} = {C}_{L} {P}_{lux} - {γ}_{L} {L}_{i}$$

Here the variables Pₗᵤₓ; Hᵢ; _{Ii} and Lᵢ are the activity of luxI promoter, intracellular AHL, LuxI and lysis protein of the i-th cell. Hₑ(xᵢ; t) is the extracellular concentration of AHL at the location of the i-th cell. luxI promoter is induced by AHL. b*(Iᵢ/(K_{I}+Iᵢ)) is the production term for AHL. Dₘ(Hₑ(xᵢ; t)−Hᵢ) describes the exchange of intra-and extra-celluar AHL across the cell membrane. C_{I}Pₗᵤₓ and γ_{I}Iᵢ are the production and degradation terms for LuxI. C_{L}Pₗᵤₓ and γ_{I}Iᵢ are the production and degradation terms for lysis protein. The extracellular AHL concentration Hₑ(x; t) is governed by linear diffusion equation

$$\frac{\partial {H}_{e} ( x , t )}{\partial t} = {D}_{m} ( \sum {H}_{i} δ ( x - {x}_{i} ) - {H}_{e} ( x , t ) ) - {δ}_{H} {H}_{e} ( x , t ) + {D}_{H} {∇}^{2} {H}_{e} ( x , t )$$

In the simulation, 2D finite difference methods were used to describe the diffusion of AHL. The model in traps were implemented with different side lengths (20, 40 and 60). To simulate the lysis of each cell, we assume that when the concentration of lysis protein Li is above a threshold Lₜₕ, the cell has a probability of Pᵣ=p_{L}(Lᵢ−Lₜₕ) per unit of time to lyse and once a cell lyses, it is removed from the trap.

Model parameters were chosen to qualitatively fit the experimental results and the parameters H₀;m;b; p_{L} were chosen to account for the differences of experimental measurements and dynamic behaviors between Lux-CFP and Rₚₐ-GFP strains. The parameter values for the Lux-CFP strain are α₀=0:1 (Lux promoter basal production); α_{H}=2 (Lux promoter AHL induced production); H₀=1 (AHL binding affinity to Lux promoter); m=4 (Hill coefficient of AHL induced production of Lux promoter); b=1:5 (AHL production rate); K_{I}=1 (Conc. of LuxI resulting half maximum production of AHL); Dₘ=10 (Diffusion constant of AHL across cell membrane); C_{I}=1 (LuxI copy number); γI=1 (Degradation rate of LuxI); C_{L}=1 (Lysis gene copy number); γL=0:5 (Degradation rate of lysis protein); d_{H}=0:1 (Dilution rate of extracellular AHL); D_{H}=65 (Diffusion constant of extracellular AHL); p_{L}=0:3 (Probability of lysing); Lₜₕ=1:6 (Threshold of lysis protein for lysis).

To simulate the constant-lysis Rpa-GFP strain, these parameters have different values: H₀=0:2; m=1; b=0:8; p_{L}=0:03. Besides, Rpa-GFP strain's growth rate is 10% larger than Lux-CFP strain.

### Deterministic Modeling

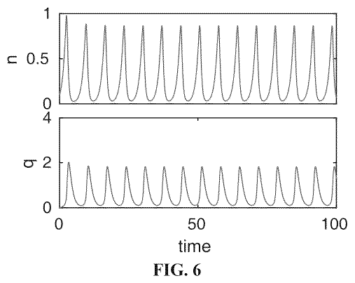

Single lysis oscillator strain: The population level mechanisms that lead to oscillations in population size as observed with the synchronized lysis circuit are described. To gain an intuitive understanding, a reduced model was used that aims to reproduce the observed population level behavior using only the fundamental ingredients of the circuit: Autocatalytic production of quorum sensing agent and quorum sensing agent-induced lysis of cells. The basic equations for a single strain equipped with the lysis circuit are as follows (see FIG. 6 for model traces):

$$\frac{dn}{dt} = α n - f ( q ) γ n \qquad ( 1 a )$$

$$\frac{dq}{dt} = [ {α}_{q} + {α}_{q}^{*} f ( q ) ] n - {γ}_{q} q \qquad ( 1 b )$$

The cell density is denoted by n. Cells divide with a rate a and die with a maximal rate γ due to lysis. 0≤f (q)≤1 characterizes the promoter under which the QS and lysis proteins are expressed, so it determines the dependence of the death rate on q and the auto-catalyzed production of the QS agent q. α_{q} is the basal production rate of QS agent, which can be increased by the presence of q to a maximum production rate of αq+αq*. q is diluted in the environment with a rate γq. A standard Hill function for f (q) was used:

$$f ( q ) = \frac{{q}^{m}}{{q}_{c}^{m} + {q}^{m}} , \qquad ( 2 )$$

where qc is the concentration of q that results in the half-maximum death rate (and auto-catalyzed production of q) and m is the Hill coefficient.

A linear stability analysis shows that the system (1) has a stable fixed point when

$$m ( 1 - \frac{α}{γ} ) < 1 + \frac{{α}_{q} γ}{{α}_{q}^{*} α} . \qquad ( 3 )$$

The border of this stability region corresponds to the onset of oscillations. Basal parameters are, unless otherwise mentioned: α=1, γ=4, α_{q}=0:4, α_{q}*=8, γ_{q}=1, q_{c}=1, m=2. These parameters lead to oscillations according to (3). All simulations are carried out using the Runge-Kutta-Fehlberg (RKF45) method. An example trajectory is depicted in FIG. 6.

While individual proteins or enzymes were not explicitly modeled, an understanding for the influence of LuxI degradation by ClpXP was gained with the model (1) using the following logic: When there is very little LuxI (i.e. the positive feedback loop has not been activated), fast degradation by ClpXP will have a strong influence on the steady-state level of LuxI. LuxI with a strong degradation tag will experience fast degradation by ClpXP leading to a low basal production rate of QS agent (α_{q}), whereas LuxI with a weak degradation tag will have a higher steady-state level and therefore a higher basal procution rate αq. In contrast, once the positive feedback has been activated, the concentration of LuxI (and consequently the parameter αq* of the model) have a much weaker dependence on its degradation tag since an abundance of LuxI produced from a fully activated promoter saturates the limited enzymatic processing capacity of ClpXP and therefore the level of LuxI will be determined mainly by dilution due to cell growth. As seen from (3), decreasing aq by a larger factor than αq* generally brings the system closer to oscillations, which is consistent with the requirement of a strong degradation tag for sustained oscillations demonstrated in FIG. 2. In summary, stronger (weaker) enzymatic degradation of LuxI was modeled by a lower (higher) value of αq.

Microfluidic traps and multiple strains: A microfluidic trap is clearly a finite environment, but because nutrients are constantly replenished by diffusion from fresh media in the channel, logistic growth (as is often assumed in other scenarios with finite carrying capacities) would be an unrealistic description of the population dynamics. Instead, it was assumed that growth is unaffected as long as the population density is below the carrying capacity c of the trap. The cell density was capped at c, corresponding to any extra cells being washed away by the flow in the main channel (“spillover”).

Numerically, the cell density was reset to c after every time step of the simulation if it exceeds c. In all simulations c=1. FIG. 6 shows that the system with standard parameters lyses just before it reaches the carrying capacity of the trap, so it is truly self-limiting. For simulations of multiple strains, two copies of the system (1) were simulated with variables {η₁,q₁} and {η₂,q₂}. Again, the system evolved freely as long as η₁+η₂<c. If η₁+η₂ exceeds c after any time step, η₁ and η₂ were set according to

$${n}_{1} = \frac{{n}_{1}^{′}}{{n}_{1}^{′} + {n}_{2}^{′}} c \qquad ( 4 )$$

$${n}_{1} = \frac{{n}_{2}^{′}}{{n}_{1}^{′} + {n}_{2}^{′}} c$$

$$if {n}_{1}^{′} + {n}_{2}^{′} > c ,$$

where η_{1′} and η_{2′} correspond to the population densities before the reset. More specifically, this way of limiting the total population density to the carrying capacity c corresponds to assuming a well-mixed environment, such that the relative population densities of the two strains remain unchanged upon spillover.

Consequently, two oscillating strains in one trap that use completely orthogonal quorum sensing systems only interact if the total population density hits the carrying capacity c. As below, the main text, the strains will eventually lock into an anti-phase pattern where they avoid reaching their peak density at the same time. In order to model cross-talk, the equation of the “receiver” strain (strain 2 in this case) was modified to read

$$\frac{{dn}_{2}}{dt} = {α}_{2} {n}_{2} - f ( {q}_{2} + ξ {q}_{1} ) {γ}_{2} {n}_{2} \qquad ( 5 a )$$

$$\frac{{dq}_{2}}{dt} = [ {α}_{q , 2} + {α}_{q , 2}^{*} f ( {q}_{2} + ξ {q}_{1} ) ] {n}_{2} - {γ}_{q , 2} {q}_{2} \qquad ( 5 b )$$

where ε determines how much strain 2 responds to the QS agent of strain 1, i.e. the strength of the cross-talk.

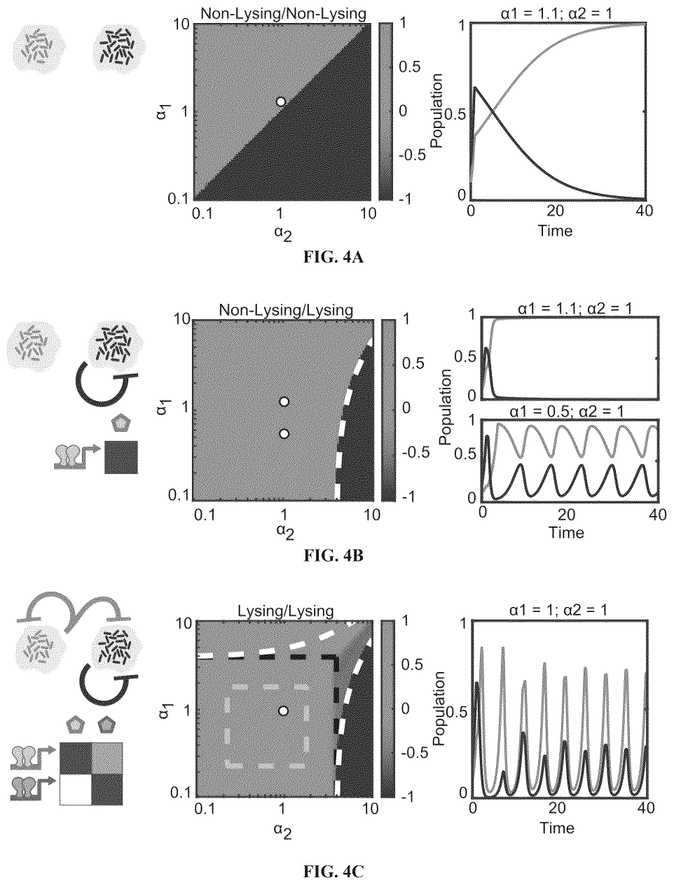

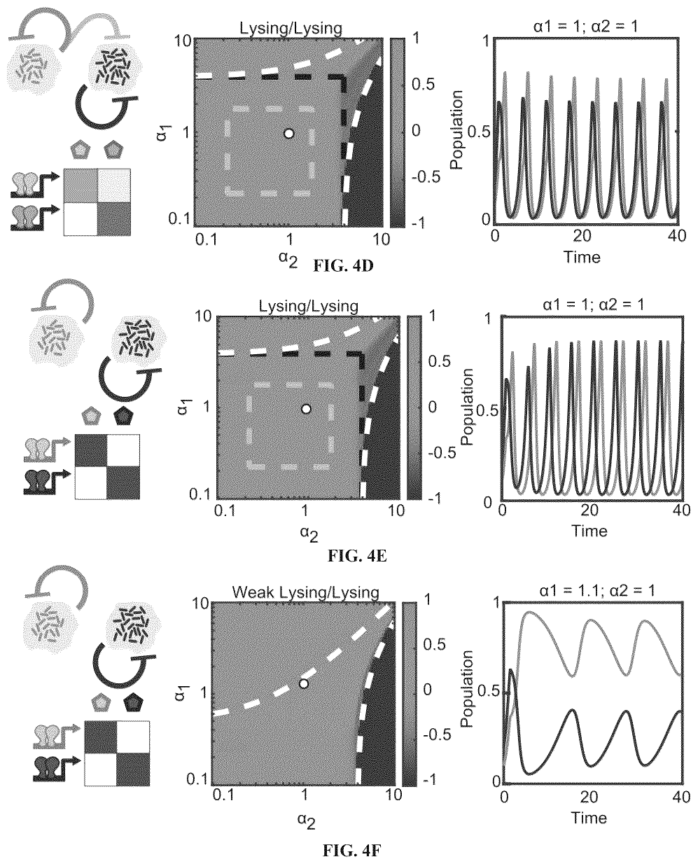

Additional parameters used in the main text: For the parameter scan of a single strain in FIG. 1, the model equations were simulated for 2000 time units for different values of the model parameter αq. The last 400 time units were used to determine the minimum, mean and maximum population density. For all parameter scans of two strains, the model equations were simulated for 500 time units and the last 100 time units were analyzed to determine the average cell densities n⁻1 and n⁻2 of the two strains. The “steady-state population ratio” shown in FIG. 4 was then calculated as (n⁻1−n⁻2)=(n⁻1+n⁻2), ranging from −1 (strain 2 dominates) to 1 (strain 1 dominates). For non-lysing strains, the model parameter qc was set to infinity. Cross-talk parameters in FIGS. 4C and 4D, are [symbol] =0:6 and [symbol] =0:12, respectively. Weak lysis (strain 1, FIG. 4F) was achieved by reducing the lysis rate of the strain to γ=0:5.

### Example 2—Communication Motifs and Quorum Sensing Signaling for Synthetic Microbial Consortia

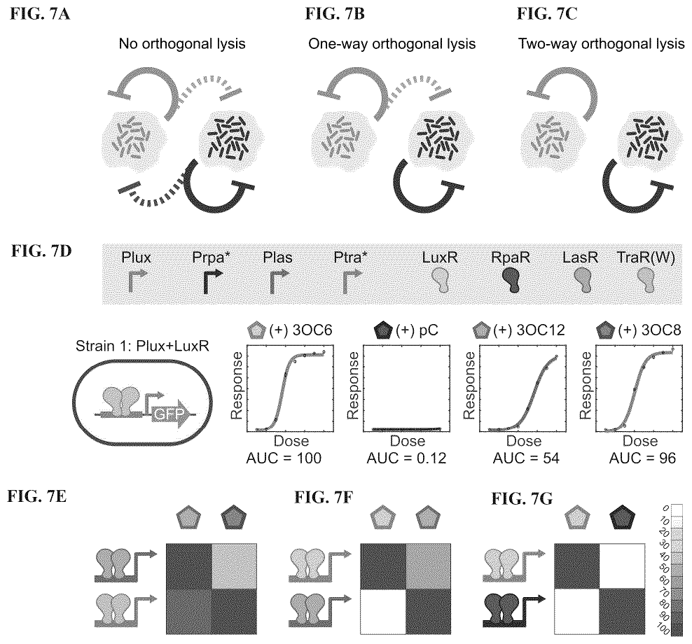

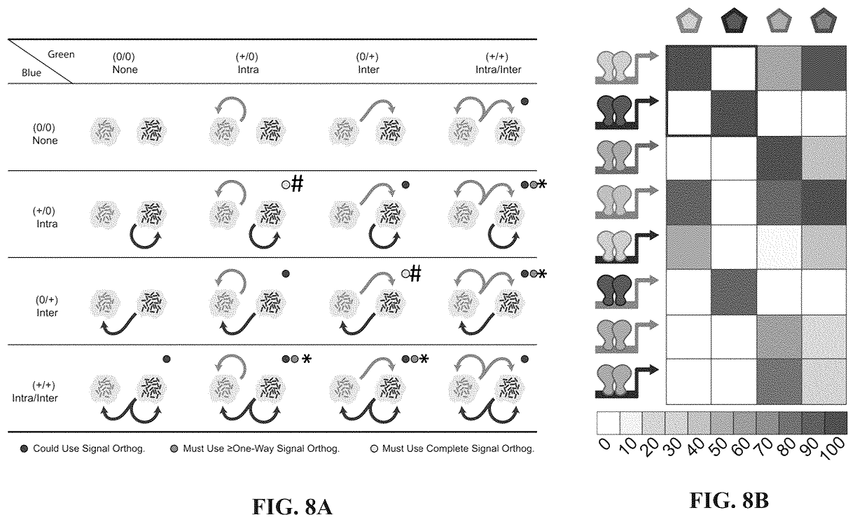

In order to engineer a stable co-culture of two competitive bacterial strains, the dynamics of a small library of quorum sensing (QS) components were first characterized (FIG. 7A-C). This was achieved by evaluating different components of natural quorum sensing systems to identify receptor-promoter pairs and signals (AHL) that yield the desired characteristic upon combination (FIG. 7D)²³. From a range of possible configurations (FIG. 8B), the Lux and Las systems were identified as suitable for one-way orthogonal signaling, and the Lux and Rpa systems were suitable for two-way orthogonal signaling. These components were used to design synchronized lysis circuits (SLCs)¹⁶ in two bacterial strains, whereby each strain is programmed to lyse upon reaching a critical population density.

To understand how an ecosystem harboring the synchronized lysis circuit (SLC) can be altered, the range of possible self-limiting dynamics of the circuit was establised (FIGS. 1A-B). The circuit exhibits oscillations, characterized by periodic lysis events, which are driven by the activation of the Lux-controlled positive feedback loop upon reaching a quorum threshold of AHL, as was seen in earlier work¹⁶. A lysis event reduces the population dramatically, and a few survivors resume the process starting again below the quorum threshold. In microfluidic devices, the fluorescent protein sfGFP reports the activation state of the circuit in this oscillatory state (FIG. 1C). A constant lysis state was discovered that is characterized by a steady state in which growth and lysis are approximately balanced, and the stable ON state of the circuit is evidenced by the constant production of sfGFP (FIG. 1D). Tuning the degradation efficiency of the activator LuxI by changing its ssrA degradation tag, bifurcation was demonstrated in lysis dynamics of the population between these two states. In a deterministic model of the circuit (FIG. 1B), lower αq corresponds to stronger enzymatic degradation of LuxI (seeMethods section for details). Consistently, the oscillatory lysis behavior was observed for the highest level of activator degradation (FIGS. 1E-F), dampened oscillations were observed at a lower level of degradation (FIGS. 1G-H), and constant lysis behavior was observed for the lowest levels of degradation (FIGS. 1I-J). The SLC therefore exhibited two main modes of dynamics lysis with respect to changes in circuit parameters.

To build a synthetic ecosystem of two orthogonal SLC strains, the previously built circuit was used based on the Lux quorum sensing system and constructed a new circuit with the Rpa system. The Rpa system had RpaR in place of LuxR and an ssrA tagged RpaI in place of LuxI (FIG. 2A). These strains are called Lux-CFP and Rpa-GFP, respectively, for convenience. Both strains' gene expression is controlled by the PluxI promoter for consistency, considering pC-bound RpaR can activate PluxI at about 90% the efficiency of AHL-bound LuxR (FIG. 8B)²³. Although these strains are in the same bacterial host, when started from equal densities in batch culture, Rpa-GFP shows a significant growth advantage over Lux-CFP (FIG. 2B). Because of this growth advantage, a 1:1 mixture of these strains in a batch culture (with or without the lysis gene), is primarily taken over by the faster growing Rpa-GFP strain by the time the strains reach stationary phase (FIG. 2C). However, if the slower growing Lux-CFP strain is enriched 100× more than the green strain, the population stabilizing effects of the lysis circuit becomes evident. Without the lysis gene, the mixture is taken over by the Lux-CFP strain, however with the lysis gene, the population ratio over the initial 10 hours keeps close to a 1:1 ratio. The ‘ortholysis’ strategy thus showed promise in batch co-culture.

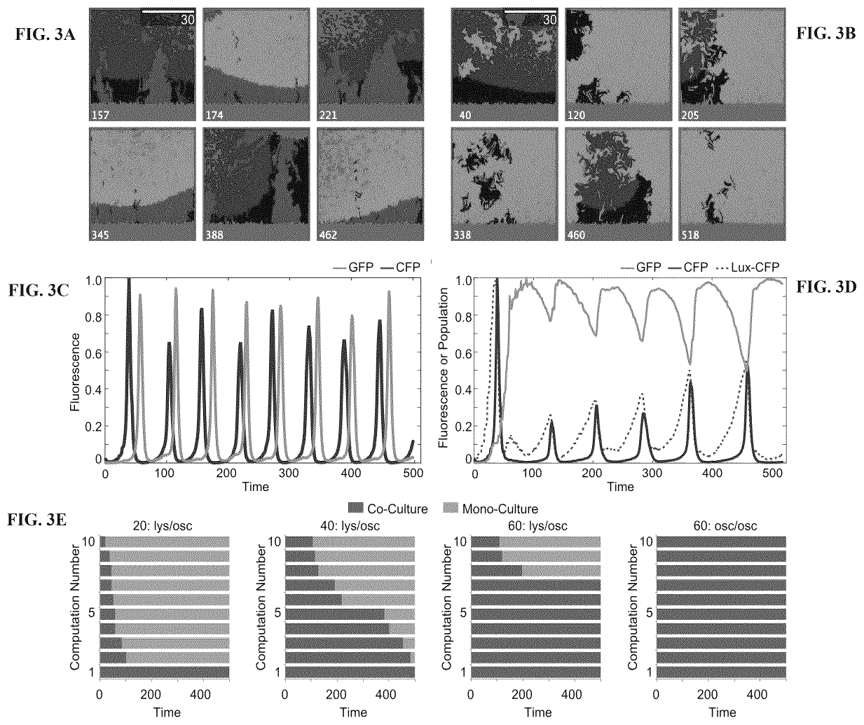

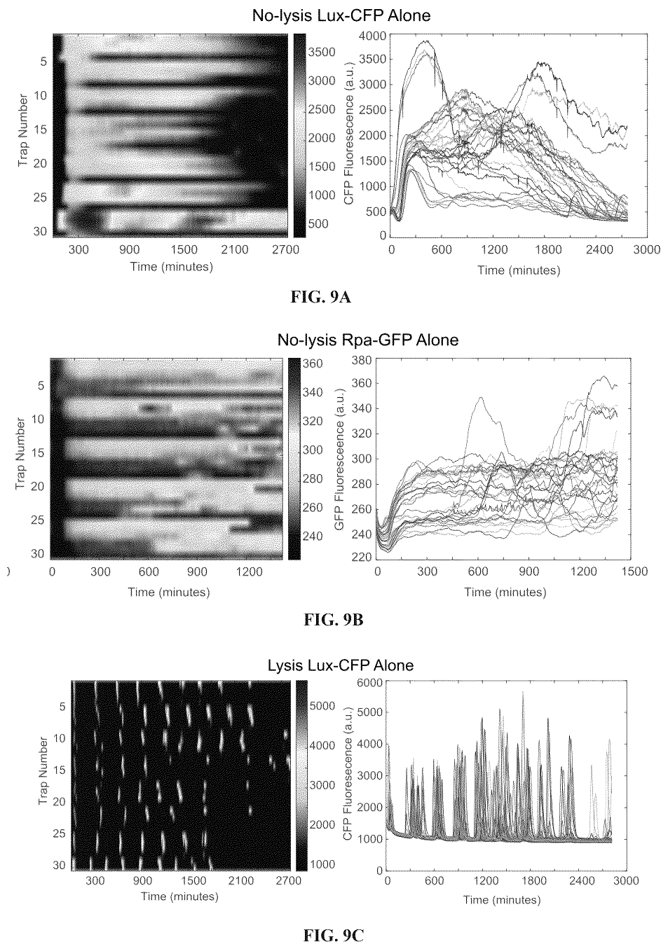

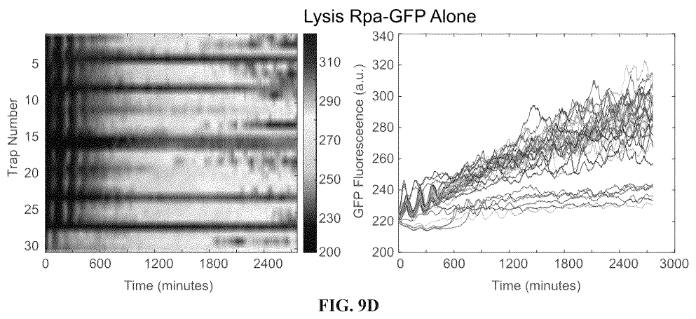

The strains were then grown in microfluidic devices, with a seeding ratio of 1:10 (Rpa-GFP to Lux-CFP) optimized for the new system, in order to examine the long-term dynamics of the co-culture. The microfluidic trap (growth chamber) harboring the two strains without the lysis gene quickly lost its co-culture and was taken over by the Rpa-GFP strain alone (FIG. 2D). This process was observed for 60 traps, and the time duration of the co-culture was measured over two days. All traps eventually lost their co-culture completely, with an average co-residence time of 6.5 hours (FIG. 2H). However, when the two orthogonal lysis strains were grown together, most of the 60 traps maintained a co-culture for the duration of the two-day experiment (FIG. 2E); all traps that lost co-culture were completely taken over by the Rpa-GFP strain. Due to differences in the inherent parameters of the two quorum sensing systems, the Rpa-GFP circuit remains in the constant lysis regime and is therefore perpetually producing sfGFP. However, the Lux-CFP strain is in the oscillatory regime and remains dark until it reaches quorum threshold and its lysis events are characterized by a punctuated burst of CFP production (FIG. 2G and FIG. 9A-D). The bimodality of the co-residence time (either lost in the first couple hours, or maintained through the end of the experiment) suggests the small volume of these reactors, and the non-deterministic loading conditions, predisposes some wells with very few Lux-CFP cells to stochastic loss of co-culture. Seemingly, depending on the environmental context, oscillatory strains are more susceptible to environmental perturbations than a strain in the constant lysis regime. However, the benefit of using a strain in the oscillatory lysis regime is that it leaves the possibility of engineering dynamic population profiles which may be useful for certain applications like timed delivery of two different payloads. Nevertheless, within the microfluidic device, the ‘ortholysis’ method is rather robust at co-culturing even competitive strains for long periods of time (FIG. 2I). Agent-based modeling was used to visually show how the ‘ortholysis’ strains might behave with different quorum sensing parameters. A system was first modeled where the quorum sensing parameters of the Rpa system were the same as the Lux system parameters used in previous studies¹⁶. However, the experimental difference in growth was used whereby the Rpa-GFP strain grows at 110% the rate of the Lux-CFP strain. With the Lux-CFP strain seeded in a 10:1 ratio to the Rpa-GFP strain in the model simulation, the resulting dynamics show anti-phase oscillations (FIG. 3A). Seemingly due to volume exclusion, as shown by their fluorescence time series, the populations enter an anti-phase pattern where the strains switch off growing and lysing (FIG. 3C). The innate differences between the two quorum sensing systems²³ were taken into consideration by changing several of the Rpa-GFP strain's quorum sensing parameters in relation to the Lux parameters used. Furthermore, based on observed phenotypic phenomenon the probability of lysing was reduced by 10-fold which allows more AHL to build up and a constant lysis dynamic to develop (FIG. 3B). The resulting dynamics were similar to the experimental observations, with a constantly lysing Rpa-GFP strain maintaining the majority of the population share, and the Lux-CFP strain intermittently firing and lysing (FIG. 3D). In order to understand how these dynamics and the size of the growth container affect stability, the agent-based model was run many times under different conditions. For conditions where Lux-CFP is oscillating and Rpa-GFP is in constant lysis (lys/osc) or where both are oscillating (osc/osc), ten simulations were done in volumes of 20, 40 and 60 each. As the size of the space increases, so does the average residence time of the co-culture (FIG. 3E), suggesting that, larger traps will have fewer issues with losing co-culture to stochastic events. As evidenced by the agent-based model, the strains demonstrate only one particular dynamic of a wide-range of possibilities facilitated by quorum sensing controlled self-lysing microbes with varying levels of orthogonality.

A reduced deterministic model was developed to explore a wider space of possible dynamics achieved through differences in growth rates, QS systems, and lysis circuit regimes. For each case, communication motifs are distinguished and suitable experimental candidate QS systems are chosen to achieve the displayed dynamic. For the two individual lysis circuits, either Non-lysing (no SLC), Lysing (SLC), or Weak Lysing (less effective SLC) were considered. With two non-lysing strains, the faster growing strain will eventually dominate the population (FIG. 4A). However, even a single strain equipped with the SLC can stabilize the co-culture, provided the non-lysising strain has the lower growth rate (FIG. 4B). In cases where both strains harbor an SLC, but there is one-way cross-talk, the strain that responds to both signals becomes entrained to the strain that only responds to its own (FIG. 4C-D). An example would be the Lux and Las systems, the Lux can respond to the Las signal, but Las is orthogonal to the Lux signal. The strength of the cross-talk determines the strength and delay of the entrainment, with strong crosstalk (FIG. 4C) exhibiting strong entrainment, and weak cross-talk (FIG. 4D) showing time-delayed entrainment. In cases where each SLC operates independently, by using signal orthogonal QS systems, the most robust co-culturing is achieved where for large ranges of growth rates, the time averaged population ratio remains around 50/50 (FIG. 4E). If one of the strains exhibits weaker lysing dynamics, in that it has a lower probability of lysing given a quorum threshold, dynamics similar to those observed in the experimental system are obtained (FIG. 2G and FIG. 4F). As seen in the experiment, the Rpa-GFP strain inhabits most of the space, with blue periodically displacing it until it reaches quorum and self-limits its population. This dynamic, as with the dynamics of each set-up, offers a distinct advantage for certain purposes. For example, a system requiring a constant production of a particular chemical and periodic bursts of a second chemical could appropriate the set-up in FIG. 4F to its advantage.

### REFERENCES

1. Gravel, D. et al. Experimental niche evolution alters the strength of the diversity-productivity relationship. Nature 469, 89-92 (2011).
2. De Roy, K. et al. Environmental conditions and community evenness determine the outcome of biological invasion. Nature communications 4, 1383 (2013).
3. Tanouchi, Y., Smith, R. P. & You, L. Engineering microbial systems to explore ecological and evolutionary dynamics. Current opinion in biotechnology 23, 791-797 (2012).
4. Wintermute, E. H. & Silver, P. A. Emergent cooperation in microbial metabolism. Molecular systems biology 6, 407 (2010).
5. Klitgord, N. & Segre, D. Environments that induce synthetic microbial ecosystems. PLoS Comput Biol 6, e1001002 (2010).
6. Little, A. E., Robinson, C. J., Peterson, S. B. & Raffa, K. F. Rules of engagement: Interspecies interactions that regulate microbial communities31. In The Social Biology of Microbial Communities: Workshop Summary, vol. 62, 375-401 (National Academies Press, 2012).
7. De Roy, K., Marzorati, M., Van den Abbeele, P., Van de Wiele, T. & Boon, N. Synthetic microbial ecosystems: an exciting tool to understand and apply microbial communities. Environmental microbiology 16, 1472-1481 (2014).
8. Gardner, T. S., Cantor, C. R. & Collins, J. J. Construction of a genetic toggle switch in escherichia coli. Nature 403, 339-342 (2000).
9. Elowitz, M. B. & Leibler, S. A synthetic oscillatory network of transcriptional regulators. Nature 403, 335-338 (2000).
10. Hasty, J., McMillen, D. & Collins, J. J. Engineered gene circuits. Nature 420, 224-230 (2002).
11. Endy, D. Foundations for engineering biology. Nature 438, 449-453 (2005).
12. You, L., Cox, R. S., Weiss, R. & Arnold, F. H. Programmed population control by cell-cell communication and regulated killing. Nature 428, 868-871 (2004).
13. Brenner, K., Karig, D. K., Weiss, R. & Arnold, F. H. Engineered bidirectional communication mediates a consensus in a microbial biofilm consortium. Proceedings of the National Academy of Sciences 104, 17300-17304 (2007).
14. Danino, T., Mondragón-Palomino, O., Tsimring, L. & Hasty, J. A synchronized quorum of genetic clocks. Nature 463, 326-330 (2010).
15. Chen, Y., Kim, J. K., Hirning, A. J., Josic, K. & Bennett, M. R. Emergent genetic oscillations in a synthetic microbial consortium. Science 349, 986-989 (2015).
16. Din, M. O. et al. Synchronized cycles of bacterial lysis for in vivo delivery. Nature 536, 81-85 (2016).
17. Fulget, N., Poughon, L., Richalet, J. & Lasseur, C. Melissa: global control strategy of the artificial ecosystem by using first principles models of the compartments. Advances in Space Research 24, 397-405 (1999).
18. Chen, Y. Development and application of co-culture for ethanol production by co-fermentation of glucose and xylose: a systematic review. Journal of industrial microbiology & biotechnology 38, 581-597 (2011).
19. Dejonghe, W. et al. Synergistic degradation of linuron by a bacterial consortium and isolation of a single linuron-degrading variovorax strain. Applied and Environmental Microbiology 69, 1532-1541 (2003).
20. Petrof, E. O. et al. Stool substitute transplant therapy for the eradication of clostridium difficile infection: “repoopulating” the gut. Microbiome 1, 1 (2013).
21. Gause, G. F. The struggle for existence (Courier Corporation, 2003).
22. Faust, K. & Raes, J. Microbial interactions: from networks to models. Nature Reviews Microbiology 10, 538-550 (2012).
23. Scott, S. R. & Hasty, J. Quorum sensing communication modules for microbial consortia. ACS synthetic biology (2016).
24. Renda, B. A., Hammerling, M. J. & Barrick, J. E. Engineering reduced evolutionary potential for synthetic biology. Molecular BioSystems 10, 1668-1678 (2014).
25. Danino, T. et al. Programmable probiotics for detection of cancer in urine. Science translational medicine 7, 289ra84-289ra84 (2015).
26. Bittihn, P., Hasty, J. & Tsimring, L. S. Suppression of beneficial mutations in dynamic microbial populations. Physical Review Letters 118, 028102 (2017).
27. Balagaddé, F. K. et al. A synthetic escherichia coli predator-prey ecosystem. Molecular systems biology 4, 187 (2008).
28. Shou, W., Ram, S. & Vilar, J. M. Synthetic cooperation in engineered yeast populations. Proceedings of the National Academy of Sciences 104, 1877-1882 (2007).
29. Foster, K. R. & Bell, T. Competition, not cooperation, dominates interactions among culturable microbial species. Current biology 22, 1845-1850 (2012).
30. Zemke, A. C. & Bomberger, J. M. Microbiology: Social suicide for a good cause. Current Biology 26, R80-R82 (2016).
31. Prindle, A. et al. A sensing array of radically coupled genetic/biopixels/'. Nature 481, 39-44 (2012).
32. Pédelacq, J.-D., Cabantous, S., Tran, T., Terwilliger, T. C. & Waldo, G. S. Engineering and characterization of a superfolder green fluorescent protein. Nature biotechnology 24, 79-88 (2006).
33. Lutz, R. & Bujard, H. Independent and tight regulation of transcriptional units in Escherichia coli via the lacr/o, the tetr/o and arac/i1-i2 regulatory elements. Nucleic acids research 25, 1203-1210 (1997).
34. Marguet, P., Tanouchi, Y., Spitz, E., Smith, C. & You, L. Oscillations by minimal bacterial suicide circuits reveal hidden facets of host-circuit physiology. PloS one 5, e11909 (2010).
35. Quan, J. & Tian, J. Circular polymerase extension cloning of complex gene libraries and pathways. PloS one 4, e6441 (2009).
36. Volfson, D., Cookson, S., Hasty, J. & Tsimring, L. S. Biomechanical ordering of dense cell populations. Proceedings of the National Academy of Sciences 105, 15346-15351 (2008).
37. Mather, W., Mondragón-Palomino, O., Danino, T., Hasty, J. & Tsimring, L. S. Streaming instability in growing cell populations. Physical review letters 104, 208101 (2010).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-66. (canceled)

67. A method of maintaining a co-culture by quorum sensing, the method comprising:
co-culturing at least a first bacterial strain and a second bacterial strain during a period of time of at least 12 hours; wherein:
at least one of the first and second bacterial strains has a growth advantage compared to at least one other bacterial strain; and
each of the first and second bacterial strains comprises:
a lysis plasmid having a lysis gene under the control of an activatable promoter; and
an activator plasmid having an activator gene, the expression of which promotes the accumulation of a quorum-sensing molecule,
wherein both the activatable promoter of the lysis gene and the expression of the activator gene is activated by the quorum sensing molecule,
wherein the quorum-sensing molecule of the first strain is different from the quorum-sensing molecule of the second strain, and
wherein each quorum-sensing molecule of the first and second strains has no or substantially no effect on the activatable promoter of the lysis gene of the other strain.

68. The method of claim 67, wherein the lysis plasmid and activator plasmid of at least one of the first and second strains is the same plasmid.

69. The method of claim 67, wherein the lysis plasmid and activator plasmid of at least one of the first and second strains are separate plasmids.

70. The method of claim 67, wherein the at least the first and second strains are metabolically competitive.

71. The method of claim 67, wherein the at least the first and second strains are selected from E. coli and S. typhimurium.

72. The method of claim 67, wherein the first strain does not have a growth advantage compared to the second bacterial strain.

73. The method of claim 67, wherein in each of the first and second strains the lysis plasmid comprises a lysis gene, an activatable promoter, and optionally a reporter gene, and the activator plasmid comprises an activator gene, a degradation tag, and optionally a reporter gene.

74. The method of claim 73, wherein the lysis gene in at least one of the first and second strains is E from a bacteriophage ΦX174.

75. The method of claim 73, wherein in the first strain the activatable promoter is a LuxR-AHL activatable luxI promoter and the activator gene is a LuxI.

76. The method of claim 73, wherein in the second strain the activatable promoter is a RpaR-AHL activatable RpaI promoter and the activator gene is a RpaI.

77. The method of claim 73, wherein the reporter gene is selected from a gene encoding a green fluorescent protein (GFP), cyan fluorescent protein (CFP), red fluorescent protein (RFP), or a combination thereof.

78. The method of claim 73, wherein the degradation tag is an ssrA-LAA degradation tag

79. The method of claim 67, wherein the co-culture is inoculated at a ratio of 1:100 of the bacterial strain having the growth advantage compared to the other bacterial strain.

80. The method of claim 67, wherein at least one of the plasmids is integrated into a genome of at least one of the first and second strains.

81. The method of claim 67, wherein at least one of the plasmids further comprises a plasmid-stabilizing element.

82. The method of claim 81, wherein the plasmid-stabilizing element is a toxin/antitoxin system or an actin-like protein partitioning system.

83. The method of claim 67, wherein the co-culturing occurs in a microfluidic device.

84. The method of claim 67, wherein the period of time is 12 to 72 hours.

85. The method of claim 67, wherein the period of time is selected from at least 24 hours, at least 48 hours, at least 72 hours, and at least 96 hours.

86. The method of claim 67, wherein the period of time is selected from 12 hours, 24 hours, 48 hours, 72 hours, and 96 hours.

87. The method of claim 67, wherein the co-culturing of the first and second strains is in a constant lysis state, and wherein the constant lysis state is characterized by a steady-state balance of growth and lysis of the at least two bacterial strains.

88. The method of claim 67, wherein the co-culturing of the at least two bacterial strains is oscillatory, and wherein the oscillatory co-culturing indicates a high level of activator gene degradation in at least one of the two bacterial strains.
